(19) United States
(12) Patent Application Publication
(10) Pub. No.: US 2007/0122393 A1
(43) Pub. Date: May 31, 2007

(54) Immunophenotype and immunogenicity of human adipose derived cells

(76) Inventors: Kevin R. McIntosh, Ellicott City, MD (US); James B. Mitchell II, Abingdon, MD (US); Jeffrey M. Gimble, Baton Rouge, LA (US)

Correspondence Address: Kathryn Doyle, Ph.D., J.D.;Drinker Biddle & Reath, One Logan Square, 18th & Cherry Streets, Philadelphia, PA, 19103, US

(21) Appl. No.: 11/486,637

(22) Filed: Jul. 14, 2006

Related U.S. Application Data
(60) Provisional application No. 60/699,553, filed on Jul. 15, 2005.

(51) Int. Cl. A61K 48/00; C12N 5/08

(52) U.S. Cl. 424/93.21; 435/368

(57) ABSTRACT

The present invention encompasses methods and compositions for generating an isolated adipose tissue-derived stromal cell exhibiting a low level of immunogenicity. The present invention encompasses methods and compositions for reducing an immune response associated with transplantation by administering the recipient with an amount of adipose tissue-derived stromal cells effective to reduce or inhibit host rejection and/or host versus graft disease.

### BACKGROUND OF THE INVENTION

The emerging field of regenerative medicine seeks to combine biomaterials, growth factors, and cells as novel therapeutics to repair damaged tissues and organs. As this specialty grows, there is a demand for a reliable, safe, and effective source of human adult stem cells to serve in tissue engineering applications. For regulatory purposes, these cells must be defined by quantifiable measures of purity. For practical purposes at the clinical level, these cells should be available as an “off the shelf” product immediately available upon demand at the point of care. From a commercial standpoint, the ability to use allogeneic, as opposed to autologous, adult stem cells for transplantation would have a significant positive impact on product development. Under these circumstances, a single lot of cells derived from one donor could be transplanted to multiple patients, reducing the costs of both quality control and quality assurance.

Stem cells also exist in tissues of the adult organism. The best characterized example of an adult stem cell is the hematopoietic progenitor cell isolated from the bone marrow and peripheral blood. In the absence of treatment, lethally irradiated mice died because they failed to replenish their circulating blood cells; however, transplantation of bone marrow cells from syngeneic donor animals rescued the host animal. The donor cells were responsible for repopulating the circulating blood cells. Studies have since been conducted to demonstrate that undifferentiated hematopoietic stem cells are capable of regenerating the different blood cell lineages in a host animal. These studies have provided the basis for bone marrow transplantation, a widely accepted therapeutic modality for cancer and inborn errors of metabolism.

Until recently, hematopoietic stem cells (HSC) of bone marrow origin were the only accepted “adult” stem cell capable of multipotent differentiation and self renewal. Now, evidence is accumulating to support the existence of stem cells in multiple tissue sites. These include multipotent adult progenitor cells (MAPC) mesenchymal stem cells (MSC) from the bone marrow, dermal stem cells, ear MSCs, neural stem cells from the central nervous system, hepatic and pancreatic stem cells, and stem cells from skeletal muscle. Adipose-derived stem cells (ASCs) exhibit several advantageous features. Adult stem cells derived from white adipose tissues can differentiate along the adipocyte, chondrocyte, endothelial, hematopoietic support, hepatocyte, neuronal, myogenic, and osteoblast lineage pathways in vitro (Gimble et al. 2003 Curr. Top. Dev. Biol. 58:137-60; Halvorsen et al. 2001 Metabolism 50:407-13; Halvorsen et al. 2001 Tissue Eng. 7:729-41; Hicok et al. 2004 Tissue Eng. 10:371-80; Erickson et al. 2002 Biochem. Biophys. Res. Commun. 290:763-9; Safford et al. 2004 Exp. Neurol. 187:319-28; Safford et al. 2002 Biochem. Biophys. Res. Commun. 294:371-9; Zuk et al. 2001 Tissue Eng. 7:211-28; Zuk et al. 2002 Mol. Biol. Cell. 13:4279-95; Mizuno et al. 2003 J. Nippon Med. Sch. 70:300-6; Seo et al. 2005 Biochem. Biophys. Res. Commun. 328:258-64). Adipose tissue is accessible, abundant, and replenishable, thereby providing a potential adult stem cell reservoir for each individual. These findings represent the work of many groups working independently. However, the cell preparations in different laboratories are not identical. It is believed that these independent groups begin their cell isolation procedures by subjecting the minced adipose tissue to a collagenase digestion followed by a centrifugation step. The initial cell pellet is identified as the “stromal vascular fraction” (SVF). Some groups have focused their attention exclusively on this minimally processed cell population. Others expand the plastic adherent subpopulation of the SVF cells for multiple passages; these are the cells that have been identified as ASCs.

The mammalian immune system plays a central role in protecting individuals from infectious agents and preventing tumor growth. However, the same immune system can produce undesirable effects such as the rejection of cell, tissue and organ transplants from unrelated donors. The immune system does not distinguish beneficial intruders, such as a transplanted tissue, from those that are harmful, and thus the immune system rejects transplanted tissues or organs. Rejection of transplanted organs is generally mediated by alloreactive T cells present in the host which recognize donor alloantigens or xenoantigens.

The transplantation of cells, tissues, and organs between genetically disparate individuals invariably results in the risk of graft rejection. Nearly all cells express products of the major histocompatibility complex, MHC class I molecules. Further, many cell types can be induced to express MHC class II molecules when exposed to inflammatory cytokines. Additional immunogenic molecules include those derived from minor histocompatibility antigens such as Y chromosome antigens recognized by female recipients. Rejection of allografts is mediated primarily by T cells of both the CD4 and CD8 subclasses (Rosenberg et al., 1992 Annu. Rev. Immunol. 10:333). Alloreactive CD4+ T cells produce cytokines that exacerbate the cytolytic CD8 response to alloantigen. Within these subclasses, competing subpopulations of cells develop after antigen stimulation that are characterized by the cytokines they produce. Th1 cells, which produce IL-2 and IFN-γ, are primarily involved in allograft rejection (Mossmann et al., 1989 Annu. Rev. Immunol. 7:145). Th2 cells, which produce IL-4 and IL-10, can down-regulate Th1 responses through IL-10 (Fiorentino et., 1989 J. Exp. Med. 170:2081). Indeed, much effort has been expended to divert undesirable Th1 responses toward the Th2 pathway. Undesirable alloreactive T cell responses in patients (allograft rejection, graft versus host disease) are typically treated with immunosuppressive drugs such as prednisone, azathioprine, and cyclosporine A. Unfortunately, these drugs generally need to be maintained for the life of the patient and they have a multitude of dangerous side effects including generalized immunosuppression. A much better approach than pan immunosuppression is to induce specific or localized suppression to donor cell alloantigens, leaving the remaining immune system intact.

It is believed that there are numerous ways to induce immunologic tolerance to alloantigens that would allow transplantation of allogeneic stem cells. Unfortunately, many of the approaches that have worked well in rodent animal models have not been successful when applied to nonhuman primates or humans. Similarly, the use of nuclear transfer to create clones of embryonic stem cells genetically identical to the recipient has been problematic for higher species, although limited success was recently reported for humans (Hwang et al., 2004, Science 303:1669). It is not clear how this technology could be applied to engineering other types of stem cells, and whether the time required for manipulation and expansion would obviate their usefulness.

Stem cells were reported to exhibit a low degree of immunogenicity, possibly due to their immature state of differentiation and immunoregulatory properties. Rat embryonic stem cell-like lines express low levels of MHC class I antigens and they are negative for expression of MHC class II molecules and CD80(B7-1)/86(B7-2) costimulatory molecules (Fandrich et al., 2002 Nat. Med. 8:171). These cells engrafted in the liver of immunocompetent allogeneic recipient rats when injected into the portal vein. Engraftment was attributed to lack of costimulatory molecules and the expression of FasL by the stem cell lines. Activated T cells express the Fas receptor, thus rendering them susceptible to apoptosis by the stem cell lines. Whether these properties are shared by other embryonic stem cell lines is currently unknown as transplanted fetal and embryonic stem cell-derived tissues are frequently rejected by the recipient's immune system (Bradley et al., 2002 Nat. Rev. 2:859; Kauftnan et al., 2000 E-biomed 1:11). Neural stem cells derived from rodents express low or negligible levels of MHC class I or class II antigens (McLaren et al., 2001 J. Neuroimmunol 112:35), but these cells are usually rejected after implantation into allogeneic recipients unless immunosuppressive drugs are used (Mason et al., 1986 Neuroscience 19:685; Sloan et al., 1991 Trends Neurosci. 14:341; Wood et al., 1996 Neuroscience 70:775). Rejection may be initiated after MHC molecules are up-regulated on cell membranes after exposure to inflammatory cytokines of the IFN family (McLaren et al., 2001 J. Neuroimmunol 112:35).

A major goal in organ transplantation is the permanent engraftment of the donor organ without inducing a graft rejection immune response generated by the recipient, while preserving the immunocompetence of the recipient against other foreign antigens. Typically, in order to prevent host rejection responses, nonspecific immunosuppressive agents such as cyclosporine, methotrexate, steroids and FK506 are used. These agents must be administered on a daily basis and if administration is stopped, graft rejection usually results. However, a major problem in using nonspecific immunosuppressive agents is that they function by suppressing all aspects of the immune response, thereby greatly increasing a recipient's susceptibility to infection and other diseases, including cancer. Furthermore, despite the use of immunosuppressive agents, graft rejection still remains a major source of morbidity and mortality in human organ transplantation. Most human transplants fail within 10 years without permanent graft acceptance. Only 50% of heart transplants survive 5 years and 20% of kidney transplants survive 10 years. (Opelz et al., 1981 Lancet 1:1223).

It is currently believed that a successful transplantation is dependent on the prevention and/or reduction of an unwanted immune response by the host to a transplant mediated by immune effector cells to avert host rejection of donor tissue. Also advantageous for a successful transplantation is a method to eliminate or reduce an unwanted immune response by the donor tissue against a recipient tissue known as graft versus host disease. Thus, there is long-felt need for methods to suppress or otherwise prevent an unwanted immune response associated with transplantation of cells, tissues, and organs between genetically disparate individuals. The present invention meets this need.

### BRIEF SUMMARY OF THE INVENTION

The present invention includes an isolated adipose tissue-derived adult stromal (ADAS) cell exhibiting a non-immunogenic characteristic, wherein the cell has been passaged up to at least the second passage, further wherein the cell expresses a stem cell associated characteristic selected from the group consisting of human multidrug transporter (ABCG2) and aldehyde dehydrogenase (ALDH).

In one aspect of the invention, the ADAS cell has been passaged up to at least the sixteenth passage.

In another aspect, exogenous genetic material has been introduced into the ADAS cell.

In yet another aspect, the ADAS cell is derived from a human.

In another aspect, the ADAS cell allogeneic to a recipient thereof. In yet another aspect, the ADAS cell is xenogeneic to a recipient thereof.

The invention also includes a method of treating a transplant recipient to reduce in the recipient an immune response of effector cells against an alloantigen, comprising administering to a transplant recipient, an ADAS cell exhibiting a non-immunogenic characteristic, wherein the ADAS cell has been passaged up to at least the second passage, further wherein the ADAS cell expresses a stem cell associated characteristic selected from the group consisting of human multidrug transporter (ABCG2) and aldehyde dehydrogenase (ALDH), in an amount effective to reduce an immune response of effector cells against an alloantigen, whereby in the transplant recipient, the effector cells have a reduced immune response against the alloantigen.

In one aspect, the effector cell is a T cell. In another aspect, the T cell is from a donor and the alloantigen is from a recipient. In yet another aspect, the T cell is from a recipient and the alloantigen is from a donor.

In another aspect, the T cell is present in the transplant.

In yet another aspect, the effector cell is a T cell activated prior to administration of the ADAS cell to a recipient, and further wherein the immune response is the reactivation of the T cell from the donor.

In a further aspect, the ADAS cell is administered to the transplant recipient to treat rejection of the transplant by the recipient.

In another aspect, the ADAS cell is derived from a mammal. Preferably, the mammal is a human.

In a further aspect, an immunosuppressive agent is administering to the recipient in combination with an ADAS cell.

In one aspect, the ADAS cell is administered to the recipient prior to the transplant. In another aspect, the ADAS cell is administered to the recipient concurrently with the transplant. In yet another aspect, the ADAS cell is administered as part of the transplant. In another aspect, the ADAS cell is administered to the recipient subsequent to the transplantation of the transplant.

In one aspect, the ADAS cell is administered intravenously to the recipient.

In another aspect, the effector cell is a cell of the recipient of the donor transplant.

In yet another aspect, the ADAS cell is genetically modified.

The invention also includes a method of reducing an immune response by an effector cell against an alloantigen, comprising contacting an effector cell with an ADAS cell exhibiting a non-immunogenic characteristic, wherein the ADAS cell has been passaged up to at least the second passage, further wherein the ADAS cell expresses a stem cell associated characteristic selected from the group consisting of human multidrug transporter (ABCG2) and aldehyde dehydrogenase (ALDH), in an amount effective to reduce an immune response by the effector cell against the alloantigen. Preferably, the effector cell is a T cell.

The invention also includes a method of isolating an ADAS cell from a population of cells derived from adipose tissue, the method comprising providing an antibody specific for ABCG2; contacting the population of adipose-derived cells with the antibody under conditions suitable for formation of an antibody-adipose tissue-derived stromal cell complex; and substantially separating the antibody-adipose tissue-derived stromal cell complex from the population of adipose-derived cells; thereby isolating the adipose tissue-derived stromal cell.

In one aspect, the antibody is conjugated to a physical support.

In another aspect, the physical support is selected from the group consisting of a microbead, a magnetic bead, a panning surface, a dense particle for density centrifugation, an adsorption column and an adsorption membrane.

In yet another aspect, the physical support is selected from the group consisting of a streptavidin bead and a biotin bead.

In one aspect, the antibody-adipose tissue-derived stromal cell complex is substantially separated from the population of adipose-derived cells using a method selected from the group consisting of fluorescence activated cell sorting (FACS) and magnetic activated cell sorting (MACS).

The invention also includes a method of enriching adipose tissue-derived stromal cells from a population of adipose-derived cells, the method comprising providing an antibody specific for ABCG2; contacting the population of adipose-derived cells with the antibody under conditions suitable for formation of an antibody-adipose tissue-derived stromal cell complex; and substantially separating the antibody-adipose tissue-derived stromal cell complex from the population of adipose-derived cells; thereby isolating the adipose tissue-derived stromal cell.

The invention also includes a method of identifying an ADAS cell positive for ALDH from a population of cells derived from adipose tissue, the method comprising providing a cleavable substrate specific for ALDH to the population of cells, wherein the substrate when so present in an ALDH+ cell is cleaved, further wherein the cleaved substrate emits a fluorescence thereby identifying an ALDH+ ADAS cell.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1D, is a series of images depicting Colony Forming Unit Assays (CFU) of cells derived from adipose tissue. The images depict staining profiles representative of the following colonies: (FIG. 1A) Toluidine blue⁺ CFU-F; (FIG. 1B) Alkaline phosphatase⁺ CFU-ALP; (FIG. 1C) Oil Red O⁺ CFU-Ad; and (FIG. 1D) Alizarin Red⁺ CFU-Ob.

FIG. 2 is a graph depicting a flow cytometry histogram of adipose derived cells. The flow cytometry histograms for selected hematopoietic, stem cell, and stromal cell markers from a representative donor are displayed at the stromal vascular fraction (SVF) and passage 2 (P2) stages. The percentage of cells staining positive is depicted in the upper right corner of each panel. The blue line indicates the positive staining cells while the red line indicates the isotype matched monoclonal antibody control.

FIG. 3, comprising FIGS. 3A and 3B is a series of charts demonstrating the relative change in the immunophenotype of adipose derived cells as a function of purification and passage. The percentage of positive staining cells is displayed relative to the isolation stage and passage number. FIG. 3A depicts the stromal cell associated markers CD166, CD73, CD44, and CD29. FIG. 3B depicts the stem cell associated markers human multidrug transporter (ABCG2) and CD34 (the order of the passage numbers is reversed in FIG. 3A relative to FIGS. 3B).

FIG. 4 is a chart depicting the aldehyde dehydrogenase staining of adipose derived cells as a function of purification and passage.

FIG. 5 is a graph depicting a flow cytometry histogram of adipose derived cells. The flow cytometry histograms for selected hematopoietic markers from a representative donor are displayed at the stromal vascular fraction (SVF) and passage 2 (P2) stages. The percentage of cells staining positive is depicted in the upper right corner of each panel. The blue line indicates the positive staining cells while the red line indicates the isotype matched monoclonal antibody control.

FIG. 6 is a graph depicting the immunogenicity of adipose derived cells as evaluated by mixed lymphocyte reaction (MLR) of adipose derived cells as a function of purification and passage. FIG. 5 depicts a representative MLR from a single donor. The proliferation of T cells was determined in the absence of stimulator cells, in the presence of autologous irradiated PBMCs (negative control), in the presence of allogeneic irradiated PBMCs (positive control), and in the presence of adipose derived cells (SVF, P0-P4). The stimulator cells were present at densities of 5,000, 10,000, or 20,000 per well.

FIG. 7 is a chart demonstrating the immunosuppressive effects of human adipose derived cells, including human SVF cells and ADAS cells, in a two-way mixed lymphocyte reaction.

FIG. 8 is a chart comparing the immunosuppressive effects between bone marrow stromal cells (BMSCs) and ADAS cells as measured by MLR. The difference between the ADAS and BMSC groups was not significant (p>0.05, Student's t-test).

### DETAILED DESCRIPTION

The present invention relates to the discovery that adipose tissue-derived adult stromal (ADAS) cells possess novel immunophenotypical and immunological characteristics. The novel characteristics of ADAS cells provide methods for isolating, culturing and using these cells in cell and/or gene therapy. The present invention includes compositions and methods for isolating and culturing ADAS cells as well as transplanting ADAS cells to a recipient where the likelihood of immune rejection by either the host or the graft is reduced.

The present invention is useful in transplantation of a transplant, for example a biocompatible lattice or a donor tissue, organ or cell, by reducing and/or eliminating an immune response against the transplant by the recipient's own immune system. As described more fully below, ADAS cells play a role in inhibiting and/or preventing allograft rejection of a transplant.

In addition, the disclosure provided herein demonstrates that ADAS cells are useful for the inhibition and/or prevention of an unwanted immune response by a donor transplant, for example, a biocompatible lattice or a donor tissue, organ or cell, against a recipient tissue known as graft versus host disease.

Accordingly, the present invention encompasses methods and compositions for reducing and/or eliminating an immune response to a transplant in a recipient by treating the recipient with an amount of ADAS cells effective to reduce or inhibit host rejection of the transplant. Also encompassed are methods and compositions for reducing and/or eliminating an immune response in a host by the foreign transplant against the host, i.e., graft versus host disease, by treating the donor transplant and/or recipient of the transplant ADAS cells in order to inhibit or reduce an adverse response by the donor transplant against the recipient.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “adipose tissue-derived cell” refers to a cell that originates from adipose tissue. The initial cell population isolated from adipose tissue is a heterogenous cell population including, but not limited to stromal vascular fraction (SVF) cells.

As used herein, the term “adipose derived stromal cells,” “adipose tissue-derived stromal cells,” “adipose tissue-derived adult stromal (ADAS) cells,” or “adipose-derived stem cells (ASCs)” are used interchangeably and refer to stromal cells that originate from adipose tissue which can serve as stem cell-like precursors to a variety of different cell types such as but not limited to adipocytes, osteocytes, chondrocytes, muscle and neuronal/glial cell lineages. Based on the present disclosure, ADAS cells encompass a substantially homogenous population of stem cell-like cells that possess novel immunophenotypic characteristics including but not limited to the expression of ABCG2 and ALDH. Further, the ADAS cells of the present invention are not immunogenic with respect to the elicitation of T cell proliferation. ADAS cells make up a subset population derived from adipose tissue which can be separated from other components of the adipose tissue using standard culturing procedures or otherwise methods disclosed herein. In addition, ADAS cells can be isolated from a mixture of cells using the cell surface markers disclosed herein.

As used herein, the term “late passaged adipose tissue-derived stromal cell,” refers to a cell exhibiting a less immunogenic characteristic when compared to an earlier passaged cell. The immunogenicity of an adipose tissue-derived stromal cell corresponds to the number of passages. Preferably, the cell has been passaged up to at least the second passage, more preferably, the cell has been passaged up to at least the third passage, and most preferably, the cell has been passaged up to at least the fourth passage.

“Adipose” refers to any fat tissue. The adipose tissue may be brown or white adipose tissue. Preferably, the adipose tissue is subcutaneous white adipose tissue. Such cells may comprise a primary cell culture or an immortalized cell line. The adipose tissue may be from any organism having fat tissue. Preferably the adipose tissue is mammalian, most preferably the adipose tissue is human. A convenient source of human adipose tissue is that derived from liposuction surgery. However, the source of adipose tissue or the method of isolation of adipose tissue is not critical to the invention.

“Allogeneic” refers to a graft derived from a different animal of the same species.

As defined herein, an “allogeneic adipose derived adult stromal cell” is obtained from a different individual of the same species as the recipient.

“Alloantigen” is an antigen that differs from an antigen expressed by the recipient.

As used herein, the term “autologous” is meant to refer to any material derived from the same individual to which it is later to be re-introduced into the individual.

“Xenogeneic” refers to a graft derived from an animal of a different species.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures.

“Donor antigen” refers to an antigen expressed by the donor tissue to be transplanted into the recipient.

“Differentiation medium” is used herein to refer to a cell growth medium comprising an additive or a lack of an additive such that a stem cell, adipose derived adult stromal cell or other such progenitor cell, that is not fully differentiated when incubated in the medium, develops into a cell with some or all of the characteristics of a differentiated cell.

As used herein, an “effector cell” refers to a cell which mediates an immune response against an antigen. In the situation where a transplant is introduced into a recipient, the effector cells can be the recipient's own cells that elicit an immune response against an antigen present in the donor transplant. In another situation, the effector cell can be part of the transplant, whereby the introduction of the transplant into a recipient results in the effector cells present in the transplant eliciting an immune response against the recipient of the transplant.

“Expandability” is used herein to refer to the capacity of a cell to proliferate, for example, to expand in number or in the case of a cell population to undergo population doublings.

“Graft” refers to a cell, tissue, organ or otherwise any biological compatible lattice for transplantation.

By “growth factors” is intended the following specific factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), and bone morphogenetic protein at concentrations of between picogram/ml to milligram/ml levels.

As used herein, the term “growth medium” is meant to refer to a culture medium that promotes growth of cells. A growth medium will generally contain animal serum. In some instances, the growth medium may not contain animal serum.

“Immunophenotype” of a cell is used herein to refer to the phenotype of a cell in terms of the surface protein profile of a cell.

An “isolated cell” refers to a cell which has been separated from other components and/or cells which naturally accompany the isolated cell in a tissue or mammal.

As used herein, the term “multipotential” or “multipotentiality” is meant to refer to the capability of a stem cell of the central nervous system to differentiate into more than one type of cell.

As used herein, the term “modulate” is meant to refer to any change in biological state, i.e. increasing, decreasing, and the like.

As used herein, the term “non-immunogenic” is meant to refer to the discovery that ADAS cells do not induce proliferation of T cells in an MLR. However, non-immunogenic should not be limited to T cell proliferation in an MLR, but rather should also apply to ADAS cells not inducing T cell proliferation in vivo.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

“Progression of or through the cell cycle” is used herein to refer to the process by which a cell prepares for and/or enters mitosis and/or meiosis. Progression through the cell cycle includes progression through the G1 phase, the S phase, the G2 phase, and the M-phase.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

The term “stromal cell medium” as used herein, refers to a medium useful for culturing ADAS cells. An example of a stromal cell medium is a medium comprising DMEM/F 12 Ham's, 10% fetal bovine serum, 100 U penicillin/100 μg streptomycin/0.25 μg Fungizone. Typically, the stromal cell medium comprises a base medium, serum and an antibiotic/antimycotic. However, ADAS cells can be cultured with stromal cell medium without an antibiotic/antimycotic and supplemented with at least one growth factor. Preferably the growth factor is human epidermal growth factor (hEGF). The preferred concentration of hEGF is about 1-50 ng/ml, more preferably the concentration is about 5 ng/ml. The preferred base medium is DMEM/F12 (1:1). The preferred serum is fetal bovine serum (FBS) but other sera may be used including horse serum or human serum. Preferably up to 20% FBS will be added to the above media in order to support the growth of stromal cells. However, a defined medium could be used if the necessary growth factors, cytokines, and hormones in FBS for stromal cell growth are identified and provided at appropriate concentrations in the growth medium. It is further recognized that additional components may be added to the culture medium. Such components include but are not limited to antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing stromal cells. Rather, any media capable of supporting stromal cells in tissue culture may be used.

As used herein, a “substantially purified” cell is a cell that is essentially free of other cell types. Thus, a substantially purified cell refers to a cell which has been purified from other cell types with which it is normally associated in its naturally occurring state.

“Transplant” refers to a biocompatible lattice or a donor tissue, organ or cell, to be transplanted. An example of a transplant may include but is not limited to a tissue, a stem cell, a neural stem cell, a skin cell, bone marrow, and solid organs such as heart, pancreas, kidney, lung and liver.

As used herein, a “therapeutically effective amount” is the amount of ADAS cells sufficient to provide a beneficial effect to the subject to which the cells are administered.

By the term “treating a transplant recipient to reduce in the recipient an immune response of effector cells against an alloantigen to the effector cells,” as the phrase is used herein, is meant decreasing the endogenous immune response against the alloantigen in a recipient by any method, for example administering ADAS cells to a recipient, compared with the endogenous immune response in an otherwise identical animal which was not treated with ADAS cells. The decrease in endogenous immune response can be assessed using the methods disclosed herein or any other method for assessing endogenous immune response in an animal.

As used herein “endogenous” refers to any material from or produced inside an organism, cell or system.

“Exogenous” refers to any material introduced from or produced outside an organism, cell, or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The phrase “under transcriptional control” or “operatively linked” as used herein means that the promoter is in the correct location and orientation in relation to the polynucleotides to control RNA polyrnerase initiation and expression of the polynucleotides.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (i.e., naked or contained in liposomes) and viruses that incorporate the recombinant polynucleotide.

Description

The present invention relates to the discovery that when an adipose tissue-derived adult stromal (ADAS) cell is contacted with a T cell obtained from a different individual (allogeneic T cells), the allogeneic T cell does not proliferate. Prior art dogma suggests that when T cells are mixed with any other cell type, T cell proliferation ensues. The mixed lymphocyte reaction (MLR) is a standard assay used to evaluate immunogenicity (i.e., the ability for a cell to induce T cells to proliferate as measured by MLR). The data disclosed herein demonstrate that a T cell derived from one individual is not responsive to an ADAS cell obtained from a different individual. Therefore, based upon the disclosure provided herein, an ADAS cell is not immunogenic to the immune system with respect to manifesting a T cell response.

In an embodiment of the invention, the immunophenotype and immunogenicity of an ADAS cell corresponds to the number of passages. Based on the disclosure provided herein, the later passaged cell is less immunogenic when compared to the earlier passaged cell. Preferably, the cell has been passaged for at least two passages. Preferably, the cell has been passaged for at least three passages. More preferably, the cell has been passaged for at least four passages.

In another embodiment of the invention, the cells can be cultured following isolation and, if appropriate, assayed for their immunogenicity and immunophenotype prior to therapeutic use. Preferably, the cells are cultured without differentiation using the standard cell culture media disclosed herein. Preferably, the cells can be passaged to at least five passages, and more preferably, the cells can be passaged to at least 10 passages or more. For example, the cells can be passaged to at least 15 passages, preferably at least 16 passages, more preferably at least 17 passages, yet more preferably at least 18 passages, preferably at least 19 passages or even at least 20 passages without losing their multipotentiality. Based on the disclosure presented herein, one skilled in the art would appreciate that the cells are not immunogenic and therefore are advantageous for transplantation into a mammal.

In addition to the non-immunogenic phenotype of the ADAS cell of the present invention with respect to T lymphocytes in a different individual, based on the disclosure provided herein, one skilled in the art would appreciate that an ADAS cell can suppress an MLR between allogeneic cells, for example between a T cell from one individual and a peripheral blood mononuclear cell (PBMC) from another individual. In one aspect, an ADAS cell can actively reduce the allogeneic T cell response in MLRs between a T cell and a PBMC, each obtained from different individuals.

Moreover, as discussed in more detail elsewhere herein, the immunophenotype of an ADAS cell relates to the method used in culturing the cell. For example, the immunophenotype of ADAS cells is defined as a function of, but not limited to, their stage of isolation, their passage number, whether the cells were cultured as an adherent population, and the length of time in culture. Based on the present disclosure, an ADAS cell can be successfully used in cell and/or gene therapy. That is, the cells of the present invention have a reduced likelihood of immune rejection by either the host of the graft when the cells are transplanted into an individual. In addition, an ADAS cell can be used as a therapeutic to inhibit host rejection of a transplant, and as a therapeutic to prevent or otherwise inhibit graft versus host disease following transplantation. As such, the present invention comprises compositions and methods for generating an ADAS cell useful for experimental/therapeutic purposes.

I. Isolation and Culturing of ADAS

The ADAS cells useful in the methods of the present invention may be isolated by a variety of methods known to those skilled in the art. For example, such methods are described in U.S. Pat. No. 6,153,432, which is incorporated herein in its entirety. In a preferred method, an ADAS cell is isolated from a mammalian subject, preferably a human subject.

The immunophenotype of adipose derived cells change progressively depending on culturing procedures (i.e. passage number). The adherence to plastic and subsequent expansion of human adipose-derived cells selects for a relatively homogeneous cell population, enriching for cells expressing a “stromal” immunophenotype, as compared to the heterogeneity of the crude stromal vascular fraction. ADAS cells also express stem cell associated markers including, but not limited to human multidrug transporter (ABCG2) and aldehyde dehydrogenase (ALDH).

Based on the present disclosure, the immunophentype of adipose derived cells can be exploited to serve as unique identifiers for ADAS cells. That is, the unique cell surface markers on the cells of the present invention can be used to isolate a specific sub-population of cells from a mixed population of cells derived from adipose tissue. One skilled in the art would appreciate that an antibody specific for a cell surface marker can be conjugated to a physical support (i.e. a streptavidin bead) and therefore provide the opportunity to isolate cell surface specific adipose derived cells. The isolated cell can then be cultured and expanded in vitro using methods disclosed herein or conventional methods.

A further embodiment of the present invention encompasses a method of depleting or separating a subpopulation of cells derived from adipose tissue. The invention relates to the discovery that the immunophenotype of cells derived from adipose tissue is a function of passage number. As such, a specific cell population such as ADAS cells can be depleted from such a mixed population of cells derived from adipose tissue by incubating an antibody that specifically binds to an ADAS cell within the mixed population of cells followed by a separation step including but not limited to magnetic separation. An example of an antibody that specifically binds to an ADAS cell includes, but is not limited to anti-ABCG2 antibody. The process of magnetic separation is accomplished by using magnetic beads, including but not limited to Dynabeads® (Dynal Biotech, Brown Deer, Wis.). Further to the use of Dynabeads®, MACS separation reagents (Miltenyi Biotec, Auburn, Calif.) can be used to deplete ADAS cells from a mixed population of cells. As a result of the separation step, a population of enriched ADAS cells can be obtained. Preferably, the population of ADAS cells is a purified cell population.

The immunophenotype of the cells of the invention offers a method to sort specific adipose derived cells using a flow cytometry-based cell sorter. Preferably, ADAS cells are isolated using the methods disclosed herein. The isolated ADAS cell can then be cultured in vitro to generate a desirable number of cells useful for experimental or therapeutic purposes.

Any medium capable of supporting fibroblasts in cell culture may be used to culture ADAS. Media formulations that support the growth of fibroblasts include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's salt base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non-essential amino acids), and the like. A preferred medium for culturing ADAS is DMEM, more preferably DMEM/F12 (1:1).

Additional non-limiting examples of media useful in the methods of the invention can contain fetal serum of bovine or other species at a concentration at least 1% to about 30%, preferably at least about 5% to 15%, most preferably about 10%. Embryonic extract of chicken or other species can be present at a concentration of about 1% to 30%, preferably at least about 5% to 15%, most preferably about 10%.

Following isolation, ADAS cells are incubated in stromal cell medium in a culture apparatus for a period of time or until the cells reach confluency before passing the cells to another culture apparatus. Following the initial plating, the cells can be maintained in culture for a period of about 6 days to yield the Passage 0 (P0) population. The cells can be passaged for an indefinite number of times, each passage comprising culturing the cells for about 6-7 days, during which the cell doubling times can range between 3-5 days. The culturing apparatus can be of any culture apparatus commonly used in culturing cells in vitro. A preferred culture apparatus is a culture flask with a more preferred culture apparatus being a T-225 culture flask.

ADAS cells can be cultured in stromal cell medium supplemented with hEGF in the absence of an antibiotic/antimycotic for a period of time or until the cells reach a certain level of confluence. Preferably, the level of confluence is greater than 70%. More preferably, the level of confluence is greater than 90%. A period of time can be any time suitable for the culture of cells in vitro. Stromal cell medium may be replaced during the culture of the ADAS cells at any time. Preferably, the stromal cell medium is replaced every 3 to 4 days. ADAS cells are then harvested from the culture apparatus whereupon the ADAS cells can be used immediately or cryopreserved to be stored for use at a later time. ADAS cells may be harvested by trypsinization, EDTA treatment, or any other procedure used to harvest cells from a culture apparatus.

ADAS cells described herein may be cryopreserved according to routine procedures. Preferably, about one to ten million cells are cryopreserved in stromal cell medium containing 10% DMSO in vapor phase of Liquid N₂. Frozen cells can be thawed by swirling in a 37° C. bath, resuspended in fresh growth medium, and grown as usual.

The present invention also relates to the discovery that the immunophenotype of an ADAS cell is a function of the passage number. The immunophenotype and immunogenic properties of ADAS cells are defined as a function of culturing procedures (i.e. adherence property, passage number, length of time in culture). The present disclosure demonstrates that freshly isolated stromal vascular fraction (SVF) cells and early passaged ADAS cells stimulated PBMCs, whereas later passaged ADAS cells were not immunogenic.

It was observed that human SVF cells and early passaged adherent cells derived from adipose tissue elicited a dose-dependent MLR response comparable to that of allogeneic PMBCs. With progressive passaging, the ADAS cells elicited a decreased MLR response that fell to levels comparable to those observed with autologous PBMCs by Passage 1 (P1). The cells can be passaged for an indefinite number of times. In fact, the later passaged ADAS cells are not immunogenic. For example, the cells are passaged at least to P2; more preferably, the cells are passaged at least to P3; yet more preferably, the cells are passaged at least to P4. The observed lack of immunogenic characteristics of a late passaged ADAS cell is an indication that there is a reduced likelihood of an immune rejection by either the host or the graft with respect to administering an ADAS cell to a mammal for cell/gene therapy.

Based on the present disclosure, it is also believed that later passaged cells may express immunosuppressive factors inhibiting the proliferative response of PBMCs to known stimulator cells. Therefore, the cells of the present invention can be used to induce an immunosuppressive effect in the mammal into which they are introduced. For example, when added to MLRs in the presence of allogeneic PBMCs as stimulatory cells, the later passaged cells can suppress the proliferative response.

As encompassed in the present invention, ADAS cells are typically isolated from liposuction material from a human. If the cell of the present invention is to be transplanted into a human subject, it is preferable that the ADAS cell be isolated from that same subject so as to provide for an autologous transplant. However, allogeneic transplants are also contemplated by the present invention.

Thus, in another aspect of the invention, the administered ADAS cell may be allogeneic with respect to the recipient. An allogeneic ADAS cell can be isolated from a donor that is a different individual of the same species as the recipient. Following isolation, the cell is cultured using the methods disclosed herein to produce an allogeneic product. The invention also encompasses an ADAS cell that is xenogeneic with respect to the recipient.

II. Therapy to Inhibit Host Rejection of a Transplant

The present invention includes a method of using an ADAS cell as a therapy to inhibit host rejection of a transplant. The invention is based on the discovery that ADAS cells do not stimulate allogeneic T cell proliferation. As such, the invention encompasses using ADAS cells to suppress T cell proliferation in response to transplant of exogenous organs, tissues or cells. The invention also includes a method of administering an ADAS cell to a mammal in an amount effective to reduce an immune response with respect to T cell proliferation.

One skilled in the art would appreciate, based upon the disclosure provided herein, that ADAS cells can be exploited to include suppression of T cell proliferation in response to any type of organ, tissue or cell transplanted into a mammal and obtained from a different individual. For example, the T cell proliferation in response to a cell including, but not limited to a neural stem cell (NSC), a liver cell, a cardiac cell, a chondrocyte, a kidney cell, an adipose cell, and the like, can be suppressed using ADAS cells.

The present invention encompasses a method of reducing and/or eliminating an immune response to a transplant in a recipient by administering to the recipient of the transplant an amount of ADAS cells effective to reduce or inhibit host rejection of the transplant. Without wishing to be bound to any particular theory, the ADAS cells that are administered to the recipient of the transplant inhibit the activation and proliferation of the recipient's T cells.

The transplant includes a biocompatible lattice or a donor tissue, organ or cell, to be transplanted. An example of a transplant may include, but is not limited to stem cells, skin cells or tissue, bone marrow, and solid organs such as heart, pancreas, kidney, lung and liver. Preferably, the transplant is a human NSC.

Based upon the disclosure provided herein, an ADAS cell can be obtained from any source, for example, from the tissue donor, the transplant recipient or an otherwise unrelated source (a different individual or species altogether). The ADAS cell may be autologous with respect to the T cells (obtained from the same host) or allogeneic with respect to the T cells. In the case where the ADAS cell is allogeneic, the ADAS cell may be autologous with respect to the transplant to which the T cells are responding to, or the ADAS cell may be obtained from an individual that is allogeneic with respect to both the source of the T cells and the source of the transplant to which the T cells are responding to. In addition, the ADAS cells may be xenogeneic to the T cells (obtained from an animal of a different species), for example rat ADAS cells may be used to suppress activation and proliferation of human T cells.

In a further embodiment, the ADAS cell used in the present invention can be isolated, from adipose tissue of any species of mammal, including but not limited to, human, mouse, rat, ape, gibbon, bovine. Preferably, the ADAS cell is isolated from a human, a mouse, or a rat. More preferably, the ADAS cell is isolated from a human.

Another embodiment of the present invention encompasses the route of administering ADAS cells to the recipient of the transplant. An ADAS cell can be administered by a route which is suitable for the placement of the transplant, i.e. a biocompatible lattice or a donor tissue, organ or cell, to be transplanted. An ADAS cell can be administered systemically, i.e., parenterally, by intravenous injection or can be targeted to a particular tissue or organ. An ADAS cell can be administered via a subcutaneous implantation or by injection of the cell into a connective tissue, for example, muscle.

ADAS cells can be suspended in an appropriate diluent, at a concentration of from about 0.01 to about 5×10⁶ cells/ml. Suitable excipients for injection solutions are those that are biologically and physiologically compatible with the ADAS cells and with the recipient, such as buffered saline solution or other suitable excipients. The composition for administration can be formulated, produced and stored according to standard methods complying with proper sterility and stability.

The dosage of the ADAS cells varies within wide limits and may be adjusted to the individual requirements in each particular case. The number of cells used depends on the weight and condition of the recipient, the number and/or frequency of administrations, and other variables known to those of skill in the art.

Between about 10⁵ and about 10¹³ ADAS cells per 100 kg body weight can be administered to the individual. In some embodiments, between about 1.5×10⁶ and about 1.5×10¹² cells are administered per 100 kg body weight. In some embodiments, between about 1×10⁹ and about 5×10¹¹ cells are administered per 100 kg body weight. In other embodiments, between about 4×10⁹ and about 2×10¹¹ cells are administered per 100 kg body weight. In yet other embodiments, between about 5×10⁸ cells and about 1×10¹⁰ cells are administered per 100 kg body weight.

In another embodiment of the present invention, ADAS cells are administered to the recipient prior to, or contemporaneously with a transplant to reduce and/or eliminate host rejection of the transplant. While not wishing to be bound to any particular theory, ADAS cells can be used to condition a recipient's immune system to the transplant by administering ADAS cells to the recipient, prior to, or at the same time as transplantation of the transplant, in an amount effective to reduce, inhibit or eliminate an immune response against the transplant by the recipient's T cells. The ADAS cells affect the T cells of the recipient such that the T cell response is reduced, inhibited or eliminated when presented with the transplant. Thus, host rejection of the transplant may be avoided, or the severity thereof reduced, by administering ADAS cells to the recipient, prior to, or at the same time as transplantation.

In yet another embodiment, ADAS cells can be administered to the recipient of the transplant after the administration of the transplant. Further, the present invention comprises a method of treating a patient who is undergoing an adverse immune response to a transplant by administering ADAS cells to the patient in an amount effective to reduce, inhibit or eliminate the immune response to the transplant, also known as host rejection of the transplant.

III. Therapy to Inhibit Graft Versus Host Disease Following Transplantation

The present invention includes a method of using an ADAS cell as a therapy to inhibit graft versus host disease following transplantation. The invention is based on the discovery that ADAS cells do not stimulate allogeneic T cell proliferation. It is envisioned that ADAS cells can suppress T cell proliferation in an MLR reaction. The invention also includes a method of administering an ADAS cell to a mammal in an amount effective to reduce an immune response with respect to T cell proliferation.

The present invention also provides a method of reducing and/or eliminating an immune response by a donor transplant against a recipient thereof (i.e. graft versus host reaction). Accordingly, the present invention encompasses a method of contacting a donor transplant, for example a biocompatible lattice or a donor tissue, organ or cell, preferably a neural stem cell, with ADAS cells prior to transplantation of the transplant into a recipient. The ADAS cells serve to ameliorate, inhibit or reduce an adverse response by the donor transplant against the recipient.

As discussed elsewhere herein, ADAS cells can be obtained from any source, for example, from the tissue donor, the transplant recipient or an otherwise unrelated source (a different individual or species altogether) for the use of eliminating or reducing an unwanted immune response by a transplant against a recipient of the transplant. Accordingly, ADAS cells can be autologous, allogeneic or xenogeneic to the tissue donor, the transplant recipient or an otherwise unrelated source.

In an embodiment of the present invention, the transplant is exposed to ADAS cells prior to transplantation of the transplant into the recipient. In this situation, an immune response against the transplant caused by any alloreactive recipient cell is suppressed by the ADAS cells present in the transplant. The ADAS cells are allogeneic with respect to the recipient and may be derived from the donor or from a source other than the donor or recipient. In some cases, ADAS cells autologous to the recipient may be used to suppress an immune response against the transplant. In another case, the ADAS cells may be xenogeneic with respect to the recipient, for example mouse or rat ADAS cells can be used to suppress an immune response in a human. However, it is preferable to use human ADAS cells in the present invention.

In addition to treating the transplant with ADAS cells prior to transplantation of the transplant into the recipient, the donor transplant can be

“preconditioned” or “pretreated” with cells or a tissue from the recipient prior to transplantation in order to activate T cells that may be associated with the transplant. Following the treatment of the transplant with cells or a tissue from the recipient, the cells or tissue may be removed from the transplant. The treated transplant is then further contacted with ADAS cells in order to reduce, inhibit or eliminate the activity of the T cells that were activated by the treatment of the cells or tissue from the recipient. Following this treatment of the transplant with ADAS cells, the ADAS cells may be removed from the transplant prior to transplantation into the recipient. However, some ADAS cells may adhere to the transplant, and therefore, may be introduced to the recipient with the transplant. In this situation, the ADAS cells introduced into the recipient can suppress an immune response against the recipient caused by any cell associated with the transplant. Without wishing to be bound to any particular theory, the treatment of the transplant with ADAS cells prior to transplantation of the transplant into the recipient serves to reduce, inhibit or eliminate the activity of the activated T cells, thereby preventing restimulation, or inducing hyporesponsiveness of the T cells to subsequent antigenic stimulation from a tissue and/or cells from the recipient. One skilled in the art would understand based upon the present disclosure, that preconditioning or pretreatment of the transplant prior to transplantation may reduce or eliminate the graft versus host response.

For example, in the context of bone marrow or peripheral blood stem cell (hematopoietic stem cell) transplantation, attack of the host by the graft can be reduced, inhibited or eliminated by preconditioning the donor marrow by using the pretreatment methods disclosed herein in order to reduce the immunogenicity of the graft against the recipient. As described elsewhere herein, a donor marrow can be pretreated with ADAS cells from any source, preferably with recipient ADAS cells in vitro prior to the transplantation of the donor marrow into the recipient. In a preferred embodiment, the donor marrow is first exposed to recipient tissue or cells and then treated with ADAS cells. Although not wishing to be bound to any particular theory, it is believed that the initial contact of the donor marrow with recipient tissue or cells function to activate the T cells in the donor marrow. Treatment of the donor marrow with the ADAS cells induces hyporesponsiveness or prevents restimulation of T cells to subsequent antigenic stimulation, thereby reducing, inhibiting or eliminating an adverse affect induced by the donor marrow on the recipient.

In an embodiment of the present invention, a transplant recipient suffering from graft versus host disease may be treated by administering ADAS cells to the recipient to reduce, inhibit or eliminate the severity thereof from the graft versus host disease where the ADAS cells are administered in an amount effective to reduce or eliminate graft versus host disease.

In this embodiment of the invention, preferably, the recipient's ADAS cells may be obtained from the recipient prior to the transplantation and may be stored and/or expanded in culture to provide a reserve of ADAS cells in sufficient amounts for treating an ongoing graft versus host reaction. However, as discussed elsewhere herein, ADAS cells can be obtained from any source, for example, from the tissue donor, the transplant recipient or an otherwise unrelated source (a different individual or species altogether).

IV. Advantages of Using ADAS Cells

Based upon the disclosure provided herein, it is envisioned that the ADAS cells of the present invention can be used in conjunction with current modes, for example the use of immunosuppressive drug therapy, for the treatment of host rejection to the donor tissue or graft versus host disease. An advantage of using ADAS cells in conjunction with immunosuppressive drugs in transplantation is that by using the methods of the present invention to ameliorate the severity of the immune response in a transplant recipient, the amount of immunosuppressive drug therapy used and/or the frequency of administration of immunosuppressive drug therapy can be reduced. A benefit of reducing the use of immunosuppressive drug therapy is the alleviation of general immune suppression and unwanted side effects associated with immunosuppressive drug therapy. In one embodiment, the cells of the invention is used without the requirement of immunosuppressive drug therapy.

It is also contemplated that the cells of the present invention may be administered into a recipient as a “one-time” therapy for the treatment of host rejection of donor tissue or graft versus host disease. A one-time administration of ADAS cells into the recipient of the transplant eliminates the need for chronic immunosuppressive drug therapy. However, if desired, multiple administrations of ADAS cells may also be employed.

The invention described herein also encompasses a method of preventing or treating transplant rejection and/or graft versus host disease by administering ADAS cells in a prophylactic or therapeutically effective amount for the prevention, treatment or amelioration of host rejection of the transplant and/or graft versus host disease. Based upon the present disclosure, a “therapeutic effective amount” of ADAS cells is an amount of cells that inhibit or decrease the number of activated T cells, when compared with the number of activated T cells in the absence of the administration of ADAS cells. In the situation of host rejection of the transplant, an effective amount of ADAS cells is an amount that inhibits or decreases the number of activated T cells in the recipient of the transplant when compared with the number of activated T cells in the recipient prior to administration of the ADAS cells. In the case of graft versus host disease, an effective amount of ADAS cells is an amount that inhibits or decreases the number of activated T cells present in the transplant.

An effective amount of ADAS cells can be determined by comparing the number of activated T cells in a recipient or in a transplant prior to the administration of ADAS cells thereto, with the number of activated T cells present in the recipient or transplant following the administration of ADAS cells thereto. A decrease, or the absence of an increase, in the number of activated T cells in the recipient of the transplant or in the transplant itself that is associated with the administration of ADAS cells thereto, indicates that the number of ADAS cells administered is a therapeutic effective amount of ADAS cells.

Genetic Modification

The cells of the present invention can also be used to express a foreign protein or molecule for a therapeutic purpose or in a method of tracking their assimilation and/or differentiation in the recipient. Thus, the invention encompasses expression vectors and methods for the introduction of exogenous DNA into ADAS cells with concomitant expression of the exogenous DNA in the ADAS cells. Methods for introducing and expressing DNA in a cell are well known to the skilled artisan and include those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The isolated nucleic acid can encode a molecule used to track the migration, assimilation, and survival of ADAS cells once they are introduced in the recipient. Proteins useful for tracking a cell include, but are not limited to, green fluorescent protein (GFP), any of the other fluorescent proteins (e.g., enhanced green, cyan, yellow, blue and red fluorescent proteins; Clontech, Palo Alto, Calif.), or other tag proteins (e.g., LacZ, FLAG-tag, Myc, HiS₆, and the like).

Tracking the migration, assimilation and/or differentiation of an ADAS cell of the present invention is not limited to the use of detectable molecules expressed by a vector or virus. The migration, assimilation, and/or differentiation of a cell can also be assessed using a series of probes that facilitate localization of transplanted ADAS cells within a mammal. Tracking an ADAS cell transplant may further be accomplished using antibodies or nucleic acid probes for cell-specific markers detailed elsewhere herein, such as, but not limited to, ABCG2, ALDH, and the like.

The term “genetic modification” as used herein refers to the stable or transient alteration of the genotype of an ADAS cell by intentional introduction of exogenous DNA. DNA may be synthetic, or naturally derived, and may contain genes, portions of genes, or other useful DNA sequences. The term “genetic modification” as used herein is not meant to include naturally occurring alterations such as that which occurs through natural viral activity, natural genetic recombination, or the like.

Exogenous DNA may be introduced to an ADAS cell using viral vectors (retrovirus, modified herpes viral, herpes-viral, adenovirus, adeno-associated virus, lentiviral, and the like) or by direct DNA transfection (lipofection, calcium phosphate transfection, DEAE-dextran, electroporation, and the like).

When the purpose of genetic modification of the cell is for the production of a biologically active substance, the substance will generally be one that is useful for the treatment of a given disorder. For example, it may be desired to genetically modify cells so that they secrete a certain growth factor product.

The cells of the present invention can be genetically modified by having exogenous genetic material introduced into the cells, to produce a molecule such as a trophic factor, a growth factor, a cytokine, and the like, which is beneficial to culturing the cells. In addition, by having the cells genetically modified to produce such a molecule, the cell can provide an additional therapeutic effect to the patient when transplanted into a patient in need thereof.

As used herein, the term “growth factor product” refers to a protein, peptide, mitogen, or other molecule having a growth, proliferative, differentiative, or trophic effect on a cell. For example, growth factor products useful in the treatment of CNS disorders include, but are not limited to, nerve growth factor (NGF), brain-derived neurotrophic factor (BDNF), the neurotrophins (NT-3, NT-4/NT-5), ciliary neurotrophic factor (CNTF), amphiregulin, FGF-1, FGF-2, EGF, TGFα, TGFβs, PDGF, IGFs, and the interleukins; IL-2, IL-12, IL-13.

Cells can also be modified to express a certain growth factor receptor (r) including, but not limited to, p75 low affinity NGFr, CNTFr, the trk family of neurotrophin receptors (trk, trkB, trkC), EGFr, FGFr, and amphiregulin receptors. Cells can be engineered to produce various neurotransmitters or their receptors such as serotonin, L-dopa, dopamine, norepinephrine, epinephrine, tachykinin, substance-P, endorphin, enkephalin, histamine, N-methyl D-aspartate, glycine, glutamate, GABA, ACh, and the like. Useful neurotransmitter-synthesizing genes include TH, dopa-decarboxylase (DDC), DBH, PNMT, GAD, tryptophan hydroxylase, ChAT, and histidine decarboxylase. Genes that encode various neuropeptides which may prove useful in the treatment of CNS disorders, include substance-P , neuropeptide-Y, enkephalin, vasopressin, VIP, glucagon, bombesin, cholecystokinin (CCK), somatostatin, calcitonin gene-related peptide, and the like.

The cells of the present invention can also be modified to express a cytokine. The cytokine is preferably, but not exclusively selected from the group consisting of IL-12, TNFα, IFNα, IFNβ, IFNγ, IL-7, IL-2, IL-6, IL-15, IL-21, and IL-23.

According to the present invention, gene constructs which comprise nucleotide sequences that encode heterologous proteins are introduced into the ADAS cells. That is, the cells are genetically altered to introduce a gene whose expression has therapeutic effect in the individual. According to some aspects of the invention, ADAS cells from the individual to be treated or from another individual, or from a non-human animal, may be genetically altered to replace a defective gene and/or to introduce a gene whose expression has therapeutic effect in the individual being treated.

In all cases in which a gene construct is transfected into a cell, the heterologous gene is operably linked to regulatory sequences required to achieve expression of the gene in the cell. Such regulatory sequences typically include a promoter and a polyadenylation signal.

The gene construct is preferably provided as an expression vector that includes the coding sequence for a heterologous protein operably linked to essential regulatory sequences such that when the vector is transfected into the cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof or an RNA molecule such as mRNA.

The gene construct includes the nucleotide sequence encoding the beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

The regulatory elements for gene expression include: a promoter, an initiation codon, a stop codon, and a polyadenylation signal. It is preferred that these elements be operable in the cells of the present invention. Moreover, it is preferred that these elements be operably linked to the nucleotide sequence that encodes the protein such that the nucleotide sequence can be expressed in the cells and thus the protein can be produced. Initiation codons and stop codons are generally considered to be part of a nucleotide sequence that encodes the protein. However, it is preferred that these elements are functional in the cells. Similarly, promoters and polyadenylation signals used must be functional within the cells of the present invention. Examples of promoters useful to practice the present invention include but are not limited to promoters that are active in many cells such as the cytomegalovirus promoter, SV40 promoters and retroviral promoters. Other examples of promoters useful to practice the present invention include but are not limited to tissue-specific promoters, i.e. promoters that function in some tissues but not in others; also, promoters of genes normally expressed in the cells with or without specific or general enhancer sequences. In some embodiments, promoters are used which constitutively express genes in the cells with or without enhancer sequences. Enhancer sequences are provided in such embodiments when appropriate or desirable.

The cells of the present invention can be transfected using well known techniques readily available to those having ordinary skill in the art. Exogenous genes may be introduced into the cells using standard methods where the cell expresses the protein encoded by the gene. In some embodiments, cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some embodiments, recombinant adenovirus vectors are used to introduce DNA with desired sequences into the cell. In some embodiments, recombinant retrovirus vectors are used to introduce DNA with desired sequences into the cells. In some embodiments, standard CaPO₄, DEAE dextran or lipid carrier mediated transfection techniques are employed to incorporate desired DNA into dividing cells. Standard antibiotic resistance selection techniques can be used to identify and select transfected cells. In some embodiments, DNA is introduced directly into cells by microinjection. Similarly, well-known electroporation or particle bombardment techniques can be used to introduce foreign DNA into the cells. A second gene is usually co-transfected or linked to the therapeutic gene. The second gene is frequently a selectable antibiotic-resistance gene. Transfected cells can be selected by growing the cells in an antibiotic that will kill cells that do not take up the selectable gene. In most cases where the two genes are unlinked and co-transfected, the cells that survive the antibiotic treatment have both genes in them and express both of them.

It should be understood that the methods described herein may be carried out in a number of ways and with various modifications and permutations thereof that are well known in the art. It may also be appreciated that any theories set forth as to modes of action or interactions between cell types should not be construed as limiting this invention in any manner, but are presented such that the methods of the invention can be more fully understood.

V. Transplantation

The present invention encompasses methods for administering an ADAS cell to an animal, including a human, in order to treat a disease where the introduction of new, undamaged cells will provide some form of therapeutic relief.

The skilled artisan will readily understand that ADAS cells can be transplanted into a recipient whereby upon receiving signals and cues from the surrounding milieu, the cells can further differentiate into mature cells in vivo dictated by the neighboring cellular milieu. Alternatively, the ADAS cells can be differentiated in vitro into a desired cell type and the differentiated cell can be administered to an animal in need thereof.

The invention also encompasses grafting ADAS cells in combination with other therapeutic procedures to treat disease or trauma in the body, including the CNS, skin, liver, kidney, heart, pancreas, and the like. Thus, ADAS cells can be co-grafted with other cells, both genetically modified and non-genetically modified cells which exert beneficial effects on the patient. Therefore the methods disclosed herein can be combined with other therapeutic procedures as would be understood by one skilled in the art once armed with the teachings provided herein.

The ADAS cells of this invention can be transplanted into a patient using techniques known in the art such as i.e., those described in U.S. Pat. Nos. 5,082,670 and 5,618,531, each incorporated herein by reference, or into any other suitable site in the body.

Transplantation of the cells of the present invention can be accomplished using techniques well known in the art as well as those described herein or as developed in the future. The present invention comprises a method for transplanting, grafting, infusing, or otherwise introducing the cells into a mammal, preferably, a human. Exemplified herein are methods for transplanting the cells into cardiovascular tissue of various mammals, but the present invention is not limited to such anatomical sites or to those mammals. Also, methods that relate to bone transplants are well known in the art and are described for example, in U.S. Pat. No. 4,678,470, pancreatic cell transplants are described in U.S. Pat. Nos. 6, 342,479, and 5,571,083, teaches methods for transplanting cells to any anatomical location in the body.

The cells may also be encapsulated and used to deliver biologically active molecules, according to known encapsulation technologies, including microencapsulation (see, e.g., U.S. Pat Nos. 4,352,883; 4,353,888; and 5,084,350, herein incorporated by reference), or macroencapsulation (see, e.g., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, cell number in the devices can be varied; preferably, each device contains between 10³-10⁹ cells, most preferably, about 10⁵ to 10⁷ cells. Several macroencapsulation devices may be implanted in the patient. Methods for the macroencapsulation and implantation of cells are well known in the art and are described in, for example, U.S. Pat. No. 6,498,018.

The dosage of the ADAS cells varies within wide limits and may be adjusted to the individual requirements in each particular case. The number of cells used depends on the weight and condition of the recipient, the number and/or frequency of administration, and other variables known to those of skill in the art.

The number of ADAS cells administered to a patient may be related to, for example, the cell yield after adipose tissue processing. A portion of the total number of cells may be retained for later use or cyropreserved. In addition, the dose delivered depends on the route of delivery of the cells to the patient. In one embodiment of the invention, a number of cells to be delivered to the patient is expected to be about 5.5×10⁴ cells. However, this number can be adjusted by orders of magnitude to achieve the desired therapeutic effect.

The mode of administration of the cells of the invention to the patient may vary depending on several factors including the type of disease being treated, the age of the mammal, whether the cells are differentiated or not, whether the cells have heterologous DNA introduced therein, and the like. The cells may be introduced to the desired site by direct injection, or by any other means used in the art for the introduction of compounds administered to a patient suffering from a particular disease or disorder.

The ADAS cells can be administered into a host in a wide variety of ways. Preferred modes of administration are intravascular, intracerebral, parenteral, intraperitoneal, intravenous, epidural, intraspinal, intrastemal, intra-articular, intra-synovial, intrathecal, intra-arterial, intracardiac, or intramuscular.

The ADAS cells may also be applied with additives to enhance, control, or otherwise direct the intended therapeutic effect. For example, in one embodiment, the cells may be further purified by use of antibody-mediated positive and/or negative cell selection to enrich the cell population to increase efficacy, reduce morbidity, or to facilitate ease of the procedure. Similarly, cells may be applied with a biocompatible matrix which facilitates in vivo tissue engineering by supporting and/or directing the fate of the implanted cells.

Prior to the administration of the ADAS cells into a patient, the cells may be stably or transiently transfected or transduced with a nucleic acid of interest using a plasmid, viral or alternative vector strategy. The cells may be administered following genetic manipulation such that they express gene products that intended to promote the therapeutic response(s) provided by the cells.

The use of ADAS cells for the treatment of a disease, disorder, or a condition provides an additional advantage in that the ADAS cells can be introduced into a recipient without the requirement of an immunosuppressive agent. Successful transplantation of a cell is believed to require the permanent engraftment of the donor cell without inducing a graft rejection immune response generated by the recipient. Typically, in order to prevent a host rejection response, nonspecific immunosuppressive agents such as cyclosporine, methotrexate, steroids and FK506 are used. These agents are administered on a daily basis and if administration is stopped, graft rejection usually results. However, an undesirable consequence in using nonspecific immunosuppressive agents is that they function by suppressing all aspects of the immune response (general immune suppression), thereby greatly increasing a recipient's susceptibility to infection and other diseases.

The present invention provides a method of treating a disease, disorder, or a condition by introducing ADAS cells or differentiated ADAS cells into the recipient without the requirement of immunosuppressive agents. The present invention includes the administration of an allogeneic or a xenogeneic ADAS cell, or otherwise an ADAS cell that is genetically disparate from the recipient, into a recipient to provide a benefit to the recipient. The present invention provides a method of using ADAS cells or differentiated ADAS cells to treat a disease, disorder or condition without the requirement of using immunosuppressive agents when administering the cells to a recipient. There is therefore a reduced susceptibility for the recipient of the transplanted ADAS cell or differentiated ADAS cell to incur infection and other diseases, including cancer relating conditions that is associated with immunosuppression therapy.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations which are evident as a result of the teachings provided herein.

The following experiments were performed to define the immunophenotype of human adipose derived cells, including human SVF cells and ADAS cells, at various stages of isolation, purification and expansion, using a flow cytometric based assay. In addition, the immunogenicity of the human adipose derived cells, including human SVF cells and ADAS cells, was examined in an in vitro mixed lymphocyte reaction. The results disclosed herein demonstrate that allogeneic transplantation of ADAS is feasible as a means for cell and/or gene therapy.

The results disclosed herein indicate that the isolation and expansion of ADAS cells selects for a relatively homogeneous cell population relative to the initial SVFs. The in vitro MLR assay demonstrates that it would be feasible to transplant allogeneic ADAS cells into a host and provides support for the clinical use of adult stem cells as an “off the shelf” product available to the physician and patient at the point of care.

### Example 1

### Immunophenotype of Human Adipose Derived cells: Temporal Changes in Stromal- and Stem Cell-associated Markers

Adipose tissue represents an abundant and accessible source of multipotent adult stem cells for tissue engineering applications. However, not all laboratories use cells at equivalent stages of isolation and passage. In view of the fact that some investigators use freshly isolated stromal vascular fraction (SVF) cells for tissue engineering purposes, the experiments provided herein were performed to compare the immunophenotype of human adipose derived cells, including human SVF cells and ADAS cells, as a function of adherence and passage. The immunophenotype of freshly isolated human adipose tissue-derived stromal vascular fraction cells (SVFs) was compared with serial passaged ADAS cells. The initial SVFs contained colony forming unit-fibroblasts (CFU-F) at a frequency of 1:30. Colony forming unit-adipocytes (CFU-Ad) and -osteoblasts (CFU-Ob) were present in the SVF at comparable frequencies (1:40 and 1:12, respectively). The immunophenotype of the ADAS cells based on flow cytometry changed progressively with adherence and passage. For example, stromal cell associated markers (CD13, CD29, CD44, CD63, CD73, CD90, CD166) were initially low on SVFs and increased significantly with successive passages. The stem cell associated marker CD34 was at peak levels in the SVFs and/or early passage ADAS cells and remained present, although at reduced levels, throughout the culture period. Aldehyde dehydrogenase (ALDH) and the multidrug resistance transport protein (ABCG2), both of which have been used to identify and characterize hematopoietic stem cell, were observed to be expressed by SVFs and ADAS cells at detectable levels. Endothelial cell associated markers (CD31, CD 144 or VE-cadherin, VEGF receptor 2, von Willebrand factor) were expressed on SVFs and did not change significantly with serial passage. Thus, the adherence to plastic and subsequent expansion of human ADAS cells in fetal bovine serum supplemented medium selects for a relatively homogeneous cell population, enriching for cells expressing a “stromal” immunophenotype, as compared to the heterogeneity of the crude stromal vascular fraction.

The materials and methods employed in the experiments disclosed herein are now described.

ADAS Cell Isolation and Expansion

Liposuction aspirates from subcutaneous adipose tissue sites were obtained from male and female subjects undergoing elective procedures in local plastic surgical offices. Tissues were washed 3-4 times with phosphate buffered saline (PBS) and suspended in an equal volume of PBS supplemented with 1% bovine serum and 0.1% collagenase type I prewarmed to 37° C. The tissue was placed in a shaking water bath at 37° C. with continuous agitation for 60 minutes and centrifuged for 5 minutes at 300-500 X g at room temperature. The supernatant, containing mature adipocytes, was aspirated. The pellet was identified as the stromal vascular fraction (SVF). Portions of the SVF were resuspended in cryopreservation medium (10% dimethylsulfoxide, 10% DMEM/F 12 Ham's, 80% fetal bovine serum), frozen at −80° C. in an ethanol jacketed closed container and subsequently stored in liquid nitrogen. Portions of the SVF were used in colony forming unit assays as disclosed herein. The remaining cells of the SVF were suspended and plated immediately in T225 flasks in stromal medium (DMEM/F12 Ham's, 10% fetal bovine serum (Hyclone, Logan, Utah), 100 U penicillin/100 μg streptomycin/0.25 μg Fungizone) at a density of 0.156 ml of tissue digest/square cm of surface area for expansion and culture. This initial passage of the primary cell culture was referred to as “Passage 0” (P0). Following the first 48 hours of incubation at 37° C. at 5% CO₂, the cultures were washed with PBS and maintained in stromal media until they achieved 75-90% confluence (approximately 6 days in culture). The cells were passaged by trypsin (0.05%) digestion and plated at a density of 5,000 cells/cm² (“Passage 1”). Cell viability and numbers at the time of passage were determined by trypan blue exclusion and hemacytometer cell counts. Cells were passaged repeatedly after achieving a density of 75-90% (approximately 6 days in culture) until Passage 4.

Adipogenesis

Confluent cultures of primary ADAS cells were induced to undergo adipogenesis by replacing the stromal media with adipocyte induction medium comprising DMEM/F-12 with 3% FBS, 33 μM biotin, 17 μM pantothenate, 1 μM bovine insulin, 1 μM dexamethasone, 0.25 mM isobutylmethylxanthine (IBMX), 5 μM rosiglitazone, and 100 U penicillin/100 μg streptomycin/0.25 μg Fungizone. After three days, media was changed to adipocyte maintenance medium that was identical to induction media except for the deletion of both IBMX and rosiglitazone. Cells were maintained in culture for up to nine days, with 90% of the maintenance media replaced every three days. Cultures were rinsed with PBS, fixed in formalin solution, and adipocyte differentiation was determined by staining of neutral lipids with Oil Red O.

Osteogenesis

Confluent cultures of primary ADAS cells were induced to undergo osteogenesis by replacing the stromal medium with osteogenic induction medium comprising DMEM/F-12 Ham's with 10% FBS, 10 mM γ-glycerophosphate, 50 μg/ml sodium ascorbate2-phosphate, 100 U penicillin/100 μg streptomycin/0.25 μg Fungizone. Cultures were fed with fresh osteogenic induction medium every 3-4 days for a period of up to 3 weeks. Cultures were rinsed in 0.9% NaCl, fixed in 70% ethanol, and osteogenic differentiation was determined by staining for calcium phosphate with Alizarin Red.

Colony Forming Unit (CFU) Assays

The frequency of colony forming units was determined by limiting dilution assay with the assumption that the number of progenitor cells follows a Poisson distribution (Bellows et al. 1989 Dev. Biol. 133:8-13). A portion of the SVF equivalent to 25 ml of liposuction tissue aspirate was committed to limiting dilution assays to determine the frequency of CFUs. The SVF pellet was suspended in 20 ml of PBS supplemented with 1% BSA and filtered through an autoclaved metal screen to remove large tissue fragments. A 400 μl portion of the cell suspension was removed to a 2 ml centrifuge tube, centrifuged for 3 minutes at 3,000 rpm at room temperature, and the pellet was then resuspended in 400 μl of Red Cell Lysis Buffer (Sigma, St. Louis, Mo.). After a 5 minute lysis period, a 20 μl volume of the lysate was mixed with an equal volume of trypan blue and the number of nucleated cells was determined by hemacytometer count. The remaining cells of the SVF were centrifuged at 300 X g for 5 minutes at room temperature and the resulting pellet was resuspended in stromal medium at a final concentration of 2×10⁵ cells per ml.

Four 96 well plates were prepared with 100 μl of stromal medium per well. The SVF cell suspension was serially diluted two-fold across the twelve columns of each plate, resulting in columns containing from about 10⁴ to 4 cells per well. The 96 well plates were incubated at 37° C., 5% CO₂, for nine days. At that time, one of the four plates was committed to a CFU-Fibroblast (CFU-F) assay. The plate was rinsed with PBS, fixed in formalin, stained for 20 minutes with 0. 1% toluidine blue in formalin, rinsed with water, and the number of negative wells (i.e., those that did not contain colonies of >20 toluidine blue⁺ cells) was determined for each cell concentration. This data was used to determine the number of CFU-F according to the equations F₀ =e^{-u} and u=-ln Fₒ, where Fₒ is the fraction of wells without colonies and u is the average number of precursors per well. Thus, when the fraction of wells without colonies is “0.37”, the average number of precursor cells per well is “1”.

The second plate was committed to a CFU-Alkaline Phosphatase (CFU-ALP) assay. The plate was rinsed with PBS, fixed in 100% ethanol, incubated for 1 hour in the presence of a solution comprising 36 mM sodium metaborate, 0.46 mM 5-bromo-4-chloro-3-indoxyl phosphate, 1.2 mM nitroblue tetrazolium, and 8.3 mM magnesium sulfate (pH 9.3), rinsed with water, and the number of wells that did not contain colonies of greater than 20 ALP⁺ cells was determined for each cell concentration. This data was used to determine the number of CFU-ALP according to the above formula.

The remaining two 96 well plates were induced to undergo adipogenesis and osteogenesis, respectively, as described herein. The CFU-Adipocyte (CFU-Ad) was determined by Oil Red O staining 9 days following induction. The CFU-Osteoblast (CFU-O) was determined by Alizarin Red staining >14 days following induction.

Flow Cytometry

Flow cytometry was performed on cells from the SVF as well as from cultured cells from passages 0 to 4. Cells were analyzed for phenotypic markers falling within three general categories (hematopoietic, stromal and stem cell) as well as aldehyde dehydrogenase (ALDH) activity (Stem Cell Technologies, Seattle, Wash.). The cells were analyzed using both conjugated and unconjugated mouse monoclonals. Briefly, approximately 4-8×10⁶ were acquired from each population. 1×10⁶ cells were removed for ALDH analysis and 1-2×10⁶ cells were removed for staining with the unconjugated monoclonals. 10,000 events were acquired per antibody set and a minimum of 25,000 events was acquired for the ALDH assay on a Becton Dickinson FACSCaliber flow cytometer using CELLQuest acquisition software (Becton Dickinson). Data analysis was performed using Flow Jo analysis software (Tree Star).

Conjugated Monoclonal Antibodies

The cells were washed once in flow wash buffer (1X DPBS, 0.5% BSA and 0.1% sodium azide), resuspended in blocking buffer (wash buffer with 25 μg/ml mouse IgG) and incubated for 10 minutes on ice. 100 μl of cell suspension (approximately 5×10⁵ cells) was aliquoted per tube and appropriately labeled mAbs were added for tri-color analysis (FITC, PE and APC). Appropriate isotype control combinations were performed to reflect the monoclonal isotype combinations. Antibodies directed against the following antigens (catalog #) were purchased from BD-Pharmingen unless otherwise indicated and used at the vendor recommended quantities: CD13 PE (#555394), CD29 FITC (Caltag #CD2901), CD31 FITC (Caltag #MHCD3101), CD34 PE (#348057), CD44 FITC (Cell Sciences #852.601.010), CD49a PE (#559596), CD63 FITC (#557288), CD73 PE (#550257), CD90 FITC (#555595), CD105 PE (Caltag #MHCD10504), CD144 (Chemicon #MAB1989), CD146 PE (#550315), CD166 PE (#559263), ABCG2 FITC (Chemicon #MAB4155F), VEGFr2 (Chemicon #MAB1667), and von Willebrand Factor (Chemicon MAB3442). All tubes were incubated on ice and protected from light for 30 minutes. The cells were washed once in wash buffer and fixed in 200 μl of 1% paraformaldehyde.

Unconjugated Monoclonal Antibodies

The cells were washed as stated above, blocked in wash buffer containing 5% goat serum, incubated for 10 minutes and distributed into 100 ul aliquots. The primary antibodies (CD144, anti-VEGFR2 [KDR] and anti-Von Willebrand's Factor) were added (10 μg/ml) and the cells were incubated for 30 minutes on ice. The cells were washed once in wash buffer and resuspended in wash buffer without serum. Goat anti-mouse PE-conjugated secondary antibody was added (5 μg/ml) to the suspensions containing primary antibody as well as a “secondary only” control. The cells were incubated on ice and protected from light for 15 minutes. The cells were then washed in flow wash buffer and fixed with 1% paraformaldehyde.

The results of these experiments are now described.

Cell Yield

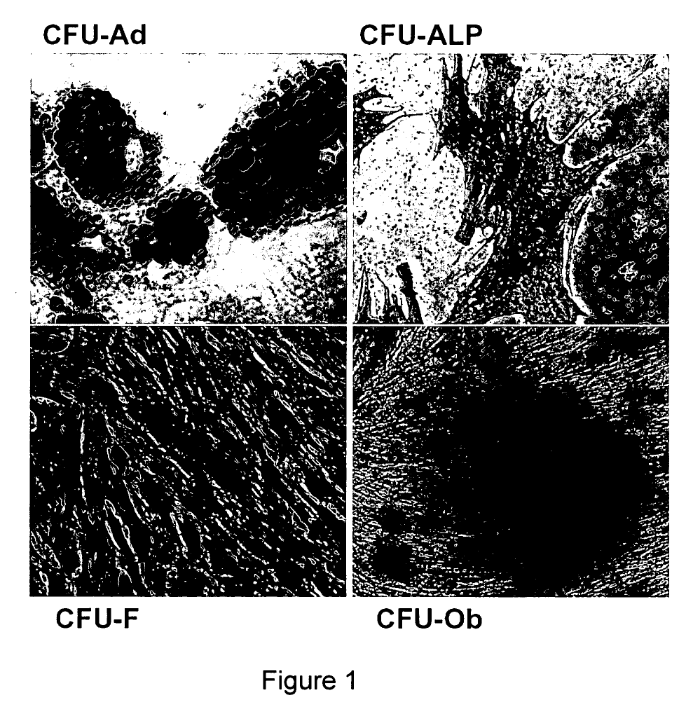

Subcutaneous adipose tissue lipoaspirates obtained from a total of 44 donors were processed by collagenase digestion and differential centrifugation. The age (mean ±S.D; 41±10 with a range of 18-64) and BMI (mean±S.D; 26.1±4.8 with a range of 19.9 to 39.2), as well as the gender distribution (84% female: 16% male) in the 44 donors were comparable to those reported in previous studies (Aust et al. 2004 Cytotherapy 6:7-14; Sen et al. 2001 J. Cell. Biochem. 81:312-9). To assess the frequency of progenitor cells in the adipose tissue, the mean number of nucleated cell number present in the stromal vascular fraction was determined as 308,849 per ml of lipoaspirate tissue (Table 1A). Based on these calculations, CFU assays were established in 96 well plates by limiting dilution assays to determine the CFU frequency for specific lineage phenotypes based on histochemical staining characteristics (Table 2). After 9 days in the culture, the number of wells containing cells staining positive for toluidine blue or alkaline phosphatase was used to determine the frequency of CFU-F and CFU-ALP, respectively (FIG. 1). At that time, identical plates were induced to undergo adipogenesis or osteogenesis. The number of wells staining positive for neutral lipids by Oil Red O or for calcium phosphate by Alizarin Red were determined after an additional 9 days or >14 days, respectively. The resulting mean CFU frequencies were as follows:

CFU-F, 1:30; CFU-ALP, 1:285; CFU-Ad, 1:40; and; CFU-Ob, 1:12 (Table 2).

TABLE 1A
Cell Yields per ml of Lipoaspirate Tissue

| Parameter | Mean ± S.D. (n) | Mean Days in Culture |
|---|---|---|
| Nucleated SVF Cells | 308,849 ± 140,354 (14) | |
| P0 Cells per cm² | 247,401 ± 136,514 (42) | 6.0 ± 2.4 |

TABLE 2
Frequency of Colony Forming Units Within the Nucleated
SVF Cell Population

| CFU Assay | Frequency (n) | Range |
|---|---|---|
| CFU-F | 1:32 ± 48 (12) | 1:5 to 1:164 |
| CFU-ALP | 1:328 ± 531 (12) | 1:11 to 1:1828 |
| CFU-Ad | 1:28 ± 49 (10) | 1:3 to 1:160 |
| CFU-Ob | 1:16 ± 22 (7) | 1:4 to 1:65 |

Following the initial plating, cells were maintained in culture for a mean period of 6 days (Table 1B) to yield the Passage 0 (P0) population. Upon harvest by trypsin digestion, a mean of 247,401 adherent P0 cells (Table 1B) were obtained per ml of original lipoaspirate tissue. These values are comparable to previous studies (Aust et al. 2004 Cytotherapy 6:7-14). Cells were passaged through an additional four successive passages of 6 to 7 days each. During each passage, the cell doubling times ranged between 3.6 to 4.7 days (Table 1B).

TABLE 1 B
Mean Cell Doubling Times and Passage Lengths

| | Mean Doubling | | |
|---|---|---|---|
| | Time (Days) ± | Mean Days in | |
| Passage | S.D. | Passage ± S.D. | N |
| P1 | 4.2 ± 2.6 | 6.3 ± 2.1 | 21 |
| P2 | 4.7 ± 2.5 | 7.0 ± 2.4 | 18 |
| P3 | 3.6 ± 0.7 | 6.1 ± 1.2 | 14 |
| P4 | 4.4 ± 2.3 | 6.6 ± 2.0 | 7 |

Immunophenotype

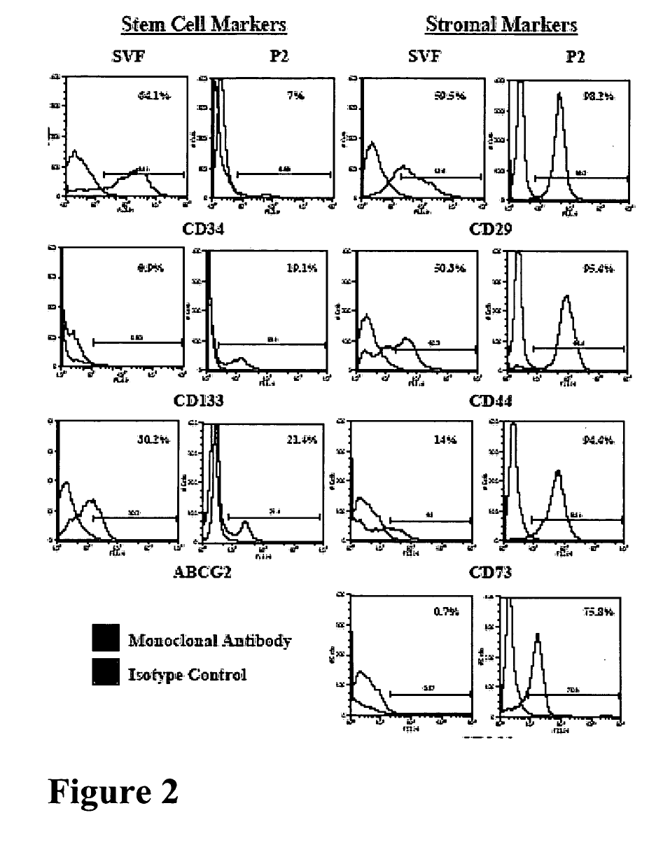

Flow cytometric analysis was performed on cells cryopreserved after each stage of purification and passage (Table 3); representative flow histograms are shown in FIG. 2. The initial SVF cells contained a subset of cells that were positive for a panel of endothelial cell-associated markers, including CD31, CD 144 (VE-cadherin), the VEGF-receptor 2, and von Willebrand factor (Table 3 and FIG. 2). The levels of these markers did not change significantly through passage 4 (P4).

TABLE 3
Phenotypic Characterization of Human Adipose-derived Cells at Progressive
Stages of Isolation and Passage 1

| Antigen | SVF (n = 7) | P0 (n = 7) | P1 (n = 7) | P2 (n = 7) | P3 (n = 7) | P4 (n = 5) |
|---|---|---|---|---|---|---|
| CD13 | 37.0 ± 0.2 | 79.5 ± | 93.0 ± | 95.5 ± | 95.9 ± | 96.8 ± 2.3 |
| | | 9.7** | 4.1*** | 2.3*** | 2.6*** | |
| CD29 | 47.7 ± 13.3 | 71.1 ± | 77.1 ± | 82.1 ± | 87.4 ± | 94.7 ± 2.05 |
| | | 30.3* | 23.6** | 21.2** | 18.8*** | |
| CD31 | 21.8 ± 10.8 | 24.4 ± 17.4 | 7.9 ± 6.0 | 7.2 ± 5.4 | 20.8 ± 14.5 | 21.0 ± 19.9 |
| CD34 | 60.0 ± 11.5 | 59.2 ± 25.4 | 21.5 ± | 5.4 ± | 2.0 ± | 1.7 ± 1.0 |
| | | | 15.1*** | 6.3*** | 2.0*** | |
| CD44 | 63.8 ± 14.5 | 84.1 ± 8.2* | 93.4 ± | 95.7 ± | 96.9 ± | 98.1 ± 1.0 |
| | | | 2.1** | 1.8*** | 3.2*** | |
| CD49a | 35.6 ± 18.6 | 28.3 | 58.8 ± | 64.0 ± | 53.4 ± 29.4 | 56.4 ± 29.3 |
| | 50.2 ± | | 29.5* | 29.1** | | |
| CD63 | 42.0 ± 7.8 | 66.1 ± 21.1 | 73.6 ± | 68.5 ± | 79.0 ± | 66.1 ± 25.1 |
| | | | 10.6** | 21.1* | 21.9** | |
| CD73 | 25.0 ± 6.2 | 74.7 ± | 85.3 ± | 89.3 ± | 93.9 ± | 94.2 ± 4.2 |
| | | 10.2*** | 37.2*** | 10.9*** | 5.5*** | |
| CD90 | 54.8 ± 10.9 | 76.6 ± 9.6* | 90.4 ± | 94.8 ± | 96.2 ± | 97.2 ± 1.0 |
| | | | 3.0*** | 1.8*** | 1.9*** | |
| CD105 | 4.9 ± 3.5 | 42.6 ± | 52.8 ± | 61.6 ± | 68.9 ± | 70.5 ± 12.1 |
| | | 17.7*** | 27.4*** | 16.6*** | 16.1*** | |
| CD1442 | 3.5 ± 1.9 | 2.7 ± 1.7 | 7.9 ± 11.6 | 4.6 ± 5.1 | 2.3 ± 0.7 | 1.8 ± 0.3 |
| CD1461 | 21.4 ± 9.3 | 29.4 ± | 19.8 ± 15.9 | 10.8 ± 4.5 | 5.1 ± 1.5 | 4.8 ± 2.8* |
| | | 10.8* | | | | |
| CD166 | 0.8 ± 0.8 | 21.7 ± | 48.5 ± | 62.8 ± | 64.1 ± | 69.2 ± 17.4 |
| | | 18.6* | 23.5** | 21.4** | 30.1** | |
| ABCG2 | 31.1 ± 15.7 | 21.5 ± 13.0 | 35.5 ± 7.6 | 19.1 ± 2.8 | 22.1 ± 12.3 | 13.9 ± 5.4 |
| ALDH3 | 14.3 ± 3 | 71.6 ± 15.6 | 79.8 ± 5.1 | 74.2 ± 3.3 | 84.6 ± 4.6 | 71.6 ± 4.8 |
| VEGFr-22 | 2.0 ± 1.6 | 2.8 ± 3.3 | 10.2 ± 13.6 | 8.9 ± 5.2 | 2.4 ± 1.9 | 1.4 ± 0.2 |
| von | 5.8 ± 1.5 | 4.6 ± 1.8 | 6.8 ± 6.2 | 6.3 ± 6.2 | 2.5 ± 1.3 | 2.0 ± 0.4 |
| Willebrand | | | | | | |
| 2 | | | | | | |

¹Data is presented as the mean ± standard deviation obtained from the number of donors indicated in parentheses.
²Data represents the mean of n = 4 donors.
³Data represents the mean of n = 3 donors.
*P value < 0.05 relative to SVF cells by Student t-test;
**P value < −0.01 relative to SVF by Student t-test:
***P value < −0.001 relative to SVF by Student t-test.

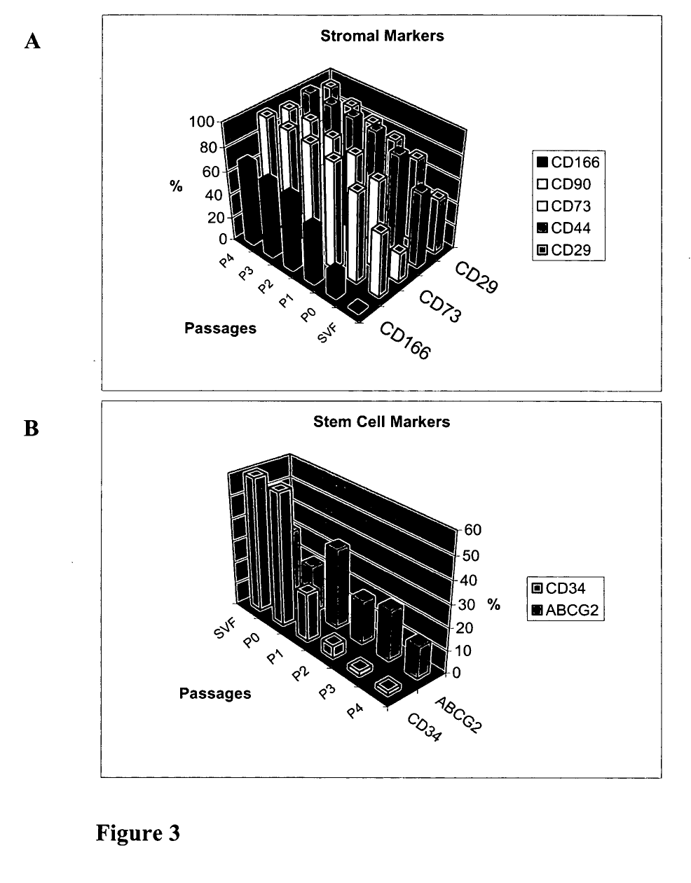

Only a subset of the initial SVF cell population expressed stromal cell-associated markers (Table 3 and FIG. 3). Less than 1% of the SVFs expressed the Activated Lymphocyte Common Adhesion Molecule (ALCAM, CD166) while 63% of the SVFs expressed the hyaluronate receptor (CD44); the levels of CD29, CD73, CD90, and CD105 were intermediate to these values. With successive passages, the percentage of cells staining positive for each of these markers increased, rising to between 69% (CD166) and 98% (CD44) by passage 4 (P4).

The initial SVF contained a subpopulation of cells positive for stem cell associated markers. A mean of 60% of the SVFs expressed the hematopoietic stem cell-associated marker CD34, a sialomucin and L-selectin ligand (Shailubhai et al., 1997 Glycobiology 7:305-14). The CD34 levels remained comparable in the P0 population and then declined significantly in successive passages (FIG. 3). The size of the CD34+ population consistently exceeded that of the hematopoietic cell population in each passage based on expression of the pan-hematopoietic marker, CD45. A mean of 31% of the SVFs displayed ABCG2, the multidrug resistance transporter responsible for the efflux of the Hoescht dye and used in the identification of the side scatter population of hematopoietic stem cells (Goodell et al., 1996 J. Exp. Med. 183:1797-806). While these levels increased during passages P0 and P1 and decreased in subsequent passages, the changes were not statistically significant relative to the SVFs.

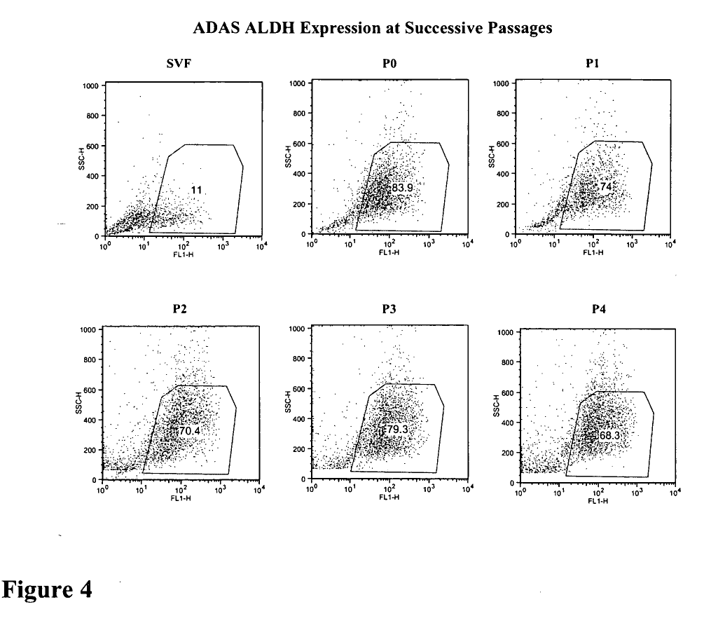

High levels of the enzyme aldehyde dehydrogenase (ALDHbr) has proven to be a novel marker for the identification and isolation of hematopoietic stem cells (Storms et al., 1999 Proc. Natl. Acad. Sci. U.S.A. 96:9118-23; Fallon et al., 2003 Br. J. Haematol. 122:99-108; Storms et al., 2005 Blood). Based on flow cytometric analysis using a fluorescent substrate, the adipose derived cells contained an ALDHbr subpopulation (Table 3, FIG. 4). While the ALDH levels were low in the SVF cells, the percentage of ALDHbr reached >70% between passages P0 to P4 with mean fluorescent intensities of 114 to 306. The percentage of ALDHbr ADAS cells fell to 10% when the cells were maintained in culture up to P9.

The results disclosed herein and from other groups demonstrate the immunophenotype of plastic adherent ADAS cells at passage 2 or later (Gronthos et al. 2001 J. Cell. Physiol. 189:54-63; Aust et al. 2004 Cytotherapy 6:7-14; Zuk et al. 2002 Mol. Biol. Cell. 13:4279-95). The ADAS cells displayed a surface protein profile that resembles that of bone marrow derived stromal cells or MSCs (Pittenger et al. 1999 Science 284:143-7) and the ADAS cells can differentiate along multiple lineage pathways (Gimble et al. 2003 Curr. Top. Dev. Biol. 58:137-60). Indeed, the ring cloning analyses of human ADAS cells have demonstrated that >50% of the clones expanded through passage 4 are capable of differentiation along two or more lineage specific pathways (Gimble et al. 2003 Curr. Top. Dev. Biol. 58:137-60). Consequently, adipose tissue presents an accessible, abundant, and alternative source of adult stem cells for potential regenerative medical applications. Studies using bone marrow MSCs isolated from 51 adult human subjects determined that the frequency of CFU-F was approximately 1:10,000 STRO-1⁺ cells (Stenderup et al., 2001 J. Bone Miner. Res. 16:1120-9). Since these authors employed an enrichment step with the STRO-1 antibody, these values are at least 3 orders of magnitude less than those currently reported for human adipose tissue. Thus, the abundance of CFU-F in adipose tissue is substantially greater than that of bone marrow.

The frequencies of CFU-Ad and CFU-Ob in adipose tissue were comparable to that of the CFU-F; however, the incidence of CFU-ALP was approximately one order of magnitude less frequent. Alkaline phosphatase enzyme activity has been used as a defining characteristic of bone marrow osteoblast progenitors and Westin-Bainton stromal cells (Friedenstein, 1968 Clin. Orthop. Relat. Res. 59:21-37; Westen et al., 1979 J. Exp. Med. 150:919-37). The current study measured alkaline phosphatase activity after 9 days in culture while alizarin red staining was performed after an additional 14 to 21 days. Since robust alkaline phosphatase staining was associated with multi-tiered cell layers (FIG. 1), it is believed that the frequency of CFU-ALP would have been closer to that of CFU-F and CFU-Ob if it had been assessed after an extended culture period.

Multiple groups have begun to isolate adipose derived cells for both in vitro and in vivo applications; however, the degree of consistency between laboratories with respect to the isolation and characterization of the cell population under investigation remains unclear. Recent studies have focused on adipose tissue derived cells at earlier stages of isolation, focusing on the SVF or adherent cells at early passage number. These cells displayed markers for the VEGF receptor, Flk-1, CD3 1, VE-cadherin, von Willebrand's factor, and other markers associated with the endothelial cell lineage. Adipose-derived SVF cells have been used to reconstitute the bone marrow of lethally irradiated mice. The SVF population has been reported to contain progenitors for macrophages and, potentially, other hematopoietic lineages. Likewise, the present disclosure indicated that the SVF cell population includes hematopoietic lineage cells based on their expression of CD11, CD14, CD45, and other markers. However, their expression is lost with progressive passage, suggesting that they do not account for the adherent cell population.

The levels of “stem cell” associated markers (CD34, ABCG2, ALDHbr) reach their peak levels in the earliest stages of culture (passages 0/1). The results presented herein demonstrate the presence of mitochondrial ALDH by tandem mass spectroscopy proteomic analysis of undifferentiated and adipocyte differentiated human ADAS cells. The percentage of ADAS cells that are ALDHbr greatly exceeds the percentage of ALDHbr cells detected in unfractionated bone marrow, which falls at or below 1% of the total cell population (Storms et al., 1996 Proc. Natl. Acad. Sci. U.S.A. 96:9118-23; Fallon et al., 2003 Br. J. Haematol. 122:99-108. Other groups have used several of these same “stem cell” associated markers (CD34 and ABCG2) in combination with CD31 to characterize and define endothelial progenitor cells in adipose derived cell populations (Miranville et al., 2004 Circulation 110:349-55). It remains to be determined if a subset of antigens or enzyme markers within this panel can be used exclusively to define stem cells derived from adipose tissue in a manner similar to that now used to characterize and isolate hematopoietic stem cells from bone marrow.

In the earliest stages of isolation, the cells of the stromal vascular fraction (SVF) exhibit low levels of “stromal” associated markers (CD13, CD29, CD44, CD73, CD90, CD105, CD166). By the later stages of culture (passages 3/4), the cells assume a more homogeneous profile with consistently high levels of “stromal” markers. Overall, this temporal expression pattern resembles that reported for human bone marrow-derived MSCs. Bone marrow MSCs progressively increased their surface expression of the markers identified as SH2 and SH3, corresponding to endoglin (CD105) and 5′-ecto nucleotidase (CD73) respectively, over 14 days of culture in vitro. By passage 4, five of the “stromal markers” (CD13, CD29, CD44, CD73, CD90) are consistently present on >90% of the ADAS cell population. Additional “stromal markers”, such as CD10, may also be of value in demonstrating the homogeneity of this population. These findings are consistent with the current immunophenotypic characterization of the adipose derived cells at various stages of isolation and expansion.

The experiments in this Example were designed to examine cells derived from human adipose tissue based on adherence characteristics and immunophenotype. It was observed that the initially isolated stromal vascular fraction cells were heterogeneous. However, only about 1 out of 30 cells actually adhered and accounted for the subsequent expansion of those cells termed adipose-derived stem cells. The frequency of adipocyte and osteoblast progenitors in the stromal vascular fraction was comparable to that of the adherent cell population. This close correlation between CFU-F, CFU-Ad, and CFU-Ob data is consistent with others demonstrating the presence of bi-F, CFU-Ad, and CFU-Ob data is consistent with others demonstrating the presence of bi-potent and tri-potent clonal cells in human adipose tissue (Zuk et al., 2002 13:4279-95). Classical “stromal” cell markers (CD13, CD29, CD44, CD73, CD90, CD105, CD166) were observed to be present on only 0.8% to 54% of the initial stromal vascular fraction cells. By late passage, stromal markers were present on up to 98% of the adipose-derived stem cell population. These temporal changes in expression resemble those reported for human bone marrow MSCs. The human ADAS cells also express stem cell associated markers such as CD34, ABCG2 and aldehyde dehydrogenase. Thus, the results presented herein demonstrate that significant changes occur in the adipose-derived cell population as a function of their isolation and culture, and have implications concerning the potential utility of human adipose tissue as a source of adult stem cells for regenerative medical therapies.

### Example 2

### The immunogenicity of Human Adipose Derived Cells

Regenerative medical techniques require an abundant source of human adult stem cells that can be readily available at the point of care. Without wishing to be bound by any particular theory, it is believed that allogeneic stem cells can achieve this goal. Since adipose tissue represents an untapped reservoir of human cells, the following experiments were designed to compared the immunogenic properties of freshly isolated human adipose tissue-derived stromal vascular fraction cells (SVFs) relative to passaged ADAS cells. The results presented herein demonstrate that the expression of hematopoietic associated markers (CD11a, CD14, CD45, CD86, HLA-DR) on adipose-derived cells decreased with passage.

In addition, it was observed that in mixed lymphocyte reactions (MLRs), SVFs and early passage ADAS cells stimulated proliferation by allogeneic responder T cells. In contrast, the ADAS cells that were passaged beyond passage P1 failed to elicit a response from T cells. Further, it was observed that late passaged ADAS cells suppressed the MLR response. Thus, the adherence to plastic and subsequent expansion of human adipose-derived cells selects for a relatively homogeneous cell population based on immunophenotype and immunogenicity. These results support the feasibility of the use of allogeneic human ADAS cell in transplantation.

The materials and methods employed in the experiments disclosed herein are now described.

BMSC Cell Isolation and Expansion

Bone marrow stromal (BMSC) cells were used in the following experiments as a control with respect to the results observed from adipose tissue-derived cells, including but not limited to SVFs and ADAS cells. Briefly, human bone marrow was purchased from Cambrex Bioscience (Walkersville, Md.) or AllCells, LLC (Berkeley, Calif.). Bone marrow aspirates were collected with heparin and fractionated over a 1.073 g/ml density gradient (Lymphocyte Separation Medium [LSM], Cambrex Bio Sciences, Walkersville, Md.) and mononuclear cells collected at the interface were plated in Dulbecco's Modified Eagles Medium—Low Glucose (HyQ DME/Low Glucose, HyClone, Logan, Utah) containing 10% FBS (JRH Biosciences, Lenexa, Kans.) that was selected based on its ability to support BMSC expansion. Nucleated cells were plated at a density of 30×10⁷ cells per T185-cm² flask. Cells were grown in primary cultures (P0) for 12 to 17 days with media changes every 3 or 4 days. When the cells became confluent, the culture was passaged using 0.05% trypsin (GIBCO, Grand Island, N.Y.) to remove adherent cells and replated as P1 cells at 1×10⁶ cells per T185-cm² flask. From this point on, the BMSCs were passaged every 7 days, with one media change every 3 to 4 days. At final harvest, BMSC were cryopreserved using a freeze solution containing 10% DMSO (Edwards Life Sciences, Irvine, Calif.) and 5% human serum albumin (JRH Biosciences) in plasmalyte (Baxter Health Care, Deerfield, Ill.). Expanded BMSCs (P2-P4) represented a homogenous population that was fibroblastic in appearance and negative for hematopoietic markers (CD45, CD14, CD3, MHC class II antigens) and positive for stromal markers (CD13, CD29, CD44, CD90, CD105). BMSCs were multipotent at P2 and P4 as shown by their ability to differentiate along the osteogenic and adipogenic lineages.

Flow Cytometry

Flow cytometry was performed as described elsewhere herein. Antibodies directed against the following antigens (catalog #) were purchased from BD-Pharmingen unless otherwise indicated and used at the vendor recommended quantities: CD11a APC (#550852), CD14 APC (#555394), CD40 APC (#555591), CD45 FITC (#555482), CD54 APC (#559771), CD80 FITC (Caltag #MHCD8001), CD86 PE (Caltag #MHCD8601), HLA-ABC APC (#555555), HLA-DR APC (#559868).

Mixed Lymphocyte Reaction (MLR)

Human Lymphocyte Populations

Peripheral blood mononuclear cells (PBMCs) were prepared by centrifugation of leukopheresed peripheral blood cells (AllCells, LLC) over an LSM density gradient. T cells were purified from a portion of the PBMCs by negative selection using magnetic beads. Briefly, PBMCs were treated with a cocktail of monoclonal antibodies (mAbs, all from Serotec, Inc., Raleigh, N.C.) chosen to bind to monocytes (anti-CD14; clone UCHM1), B cells (anti-CD19; clone LT19), natural killer cells (anti-CD56; clone ERIC-1) and cells expressing MHC class II antigens (anti-MHC class II DR; clone HL-39). PBMCs were mixed with magnetic beads coated with antimouse IgG antibody (Dynal Corp, Lake Success, N.Y.). Bead-bound cells were removed using a magnet, leaving a population of purified T cells (>90% T cells by flow cytometry using anti-CD3 mAb). Both PBMCs and purified T cells were aliquoted and cryopreserved in liquid nitrogen.

Immunogenicity Assay

The one-way MLR assay was used to determine the immunogenicity of fat-derived cell populations. The MLR was performed in 96 well microtiter plates using Iscove's Modified Dulbecco's Medium (IMDM) supplemented with sodium pyruvate, non-essential amino acids, antibiotics/antimycotics, 2-mercaptoethanol (all reagents from GIBCO, Grand Island, N.Y.) and 5% human AB serum (Pel-Freez Biologicals, Rogers, Ak.). Purified T cells derived from 2 different donors were plated at 2×10⁵ cells/donor/well. Different donors were used to maximize the chance that at least one of the T cell populations was a major mismatch to the fat-derived test cells. Stimulator cells used in the assay included autologous PBMCs (baseline response), allogeneic PBMCs (positive control response), and the test fat-derived cell populations. Stimulator cells were irradiated with 5000 rads gamma radiation delivered by a cesium irradiator prior to being added to the culture wells at various numbers, typically ranging from 5000-20,000 cells per well. Additional control cultures consisted of T cells plated in medium alone (no stimulator cells). Triplicate cultures were performed for each treatment. The cultures were incubated at 37° C. in 5% CO₂ for 6 days, pulsed with 3H-thymidine (1 μCi/well, Amersham Biosciences, Piscataway, N.J.) for 16 hours, and the cells were harvested on to glass fiber filter mats using a Skatron 96 well cell harvester (Molecular Devices Corp., Sunnyvale, N.Y.). Radioactivity incorporated into the dividing T cells deposited on the filters was determined using a scintillation counter (Microbeta Trilux Scintillation and Luminescence Counter, Wallac Inc., Gaithersburg, Md.).

Three criteria were used in assessing the immunogenicity of cell populations. These were: 1) a statistically significant difference in the T cell proliferative response (CPM) relative to that induced by autologous PBMCs (p<0.05, Student's t-test); 2) a difference of at least 750 CPM from the response induced to autologous PBMCs; and 3) a stimulation index (CPM induced by the test population divided by CPM induced by autologous PBMCs) of at least 3.0. Test populations that passed all 3 criteria were considered immunogenic.

Suppression Assay

The two-way MLR assay was used to evaluate suppression by adipose-derived cell populations. Briefly, PBMCs from two different donors were mixed in complete culture medium at 2×10⁵ cells/donor/well in 96 well microtiter plates. Fat-derived cells were added to the MLRs at 5,000, 10,000 and 20,000 cells/well. Control MLR cultures had no fat-derived cells added, or human splenic fibroblasts (CRL-7433, American Type Culture Collection, Manassas, Va.) were added at the numbers used for ADAS cells. Splenic fibroblasts were found to be the least suppressive fibroblastic cell type analyzed and were used in these experiments to define cell doses in the assay that were appropriate for calculating suppression by ADAS cells; i.e., the highest dose of splenic fibroblasts that did not mediate more than 10% suppression of the control MLR. Suppression was calculated by the following formula: Percent Suppression=(1-[Test Cell+MLR cpm÷MLR cpm])×100. Statistical significance between control and test cultures was evaluated using the Student's t-test.

The results of the experiments are now described.

Immunophenotype

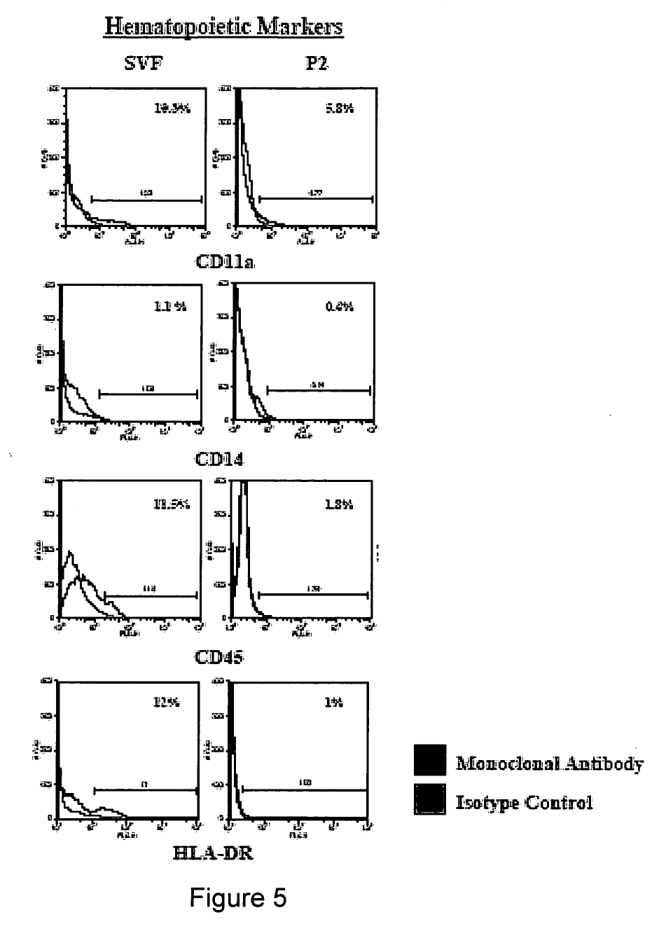

Flow cytometric analysis was performed on cells cryopreserved after each stage of purification and passage (Table 1, FIG. 5). The initial SVF and P0 cells contained a subset of cells that appeared to be monocytes since they were positive for a panel of hematopoietic markers, including the common leukocyte antigen CD45, the monocyte/macrophage markers CD11a and CD14, the MHC class II DR histocompatibility antigen and the costimulatory molecule, CD86. This population disappeared by P1 according to decreased expression for most of the aforementioned markers. The presence of monocytes in the population is significant as these cells are immunogenic and can induce a rejection response. Other hematopoietic associated markers displayed trends similar to “stromal cell” associated markers. The surface levels of CD40, CD54 (ICAM-1), and MHC class I ABC histocompatibility antigen increased significantly between the SVFs and the P3 ADAS cell populations (Table 4). The range of change varied between 1.3% to 66% for CD40 to 67% to 92% for HLA-ABC. The high level of class I antigen expression coupled with intermediate to high levels of molecules associated with costimulatory activity (CD40, CD54, CD80) would suggest that these cells could function as antigen presenting cells in the mixed lymphocyte reaction. This was investigated as described below.

TABLE 4
Phenotypic Characterization of Human Adipose-derived Cells at Progressive
Stages of Isolation and Passage¹

| Antigen | SVF (n = 7) | P0 (n= 7) | P1 (n= 7) | P2 (n= 7) | P3 (n= 7) | P4 (n= 5) |
|---|---|---|---|---|---|---|
| CD11a | 8.1 ± 3.8 | 2.2 ± 1.6** | 3.2 ± 3.0** | 1.8 ± 2.4* | 1.5 ± 1.9** | 3.1 ± 3.9 |
| CD14 | 10.1 ± 5.6 | 2.3 ± 1.7 | 0.4 ± 0.5** | 0.5 ± 1.1** | 1.0 ± 1.4* | 0.2 ± 0.2 |
| CD40 | 1.3 ± 0.7 | 14.6 ± 11.2* | 8.2 ± 8.9 | 18.6 ± 11.7* | 39.6 ± 25.2** | 65.7 ± 17.7 |
| CD45 | 17.6 ± 7.7 | 3.4 ± 2.0*** | 1.1 ± 0.9** | 0.7 ± 0.8** | 0.8 ± 0.7** | 0.9 ± 0.7 |
| CD54 | 59.9 ± 15.3 | 73.1 ± 12.9 | 76.2 ± 12.1 | 77.4 ± 8.6* | 72.1 ± 19.3 | 81.9 ± 14.1 |
| CD80 | 6.0 ± 3.9 | 6.8 ± 6.0 | 12.8 ± 9.3 | 11.9 ± 6.1 | 9.6 ± 6.4 | 6.2 ± 3.0 |
| CD86 | 10.2 ± 9.7 | 2.9 ± 2.6 | 0.5 ± 0.5 | 0.3 ± 0.3 | 0.4 ± 0.4* | 0.6 ± 0.4 |
| HLA-ABC | 66.5 ± 19.2 | 90.0 ± 7.3** | 94.0 ± 4.2** | 91.2 ± 8.7** | 90.0 ± 10.3** | 92.4 ± 6.3 |
| HLA-DR | 13.2 ± 6.8 | 4.0 ± 3.0** | 1.3 ± 0.6** | 1.9 ± 1.0** | 2.3 ± 1.4** | 2.2 ± 2.5 |

¹Data is presented as the mean ± standard deviation obtained from the number of donors indicated in parentheses.
²Data represents the mean of n = 4 donors.
*P value < 0.05 relative to SVF cells by Student's t-test;
**P value < 0.01 relative to SVF by Student's t-test:
***P value < 0.001 relative to SVF by Student's t-test.

Immunogenicity:

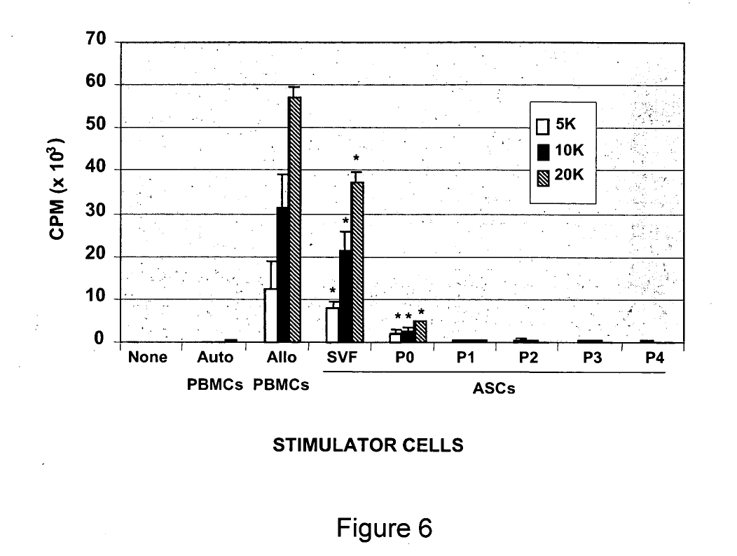

One-way MLR assays were performed to assess the immunogenicity of human adipose derived cells, including human SVF cells and ADAS cells. The proliferation of T cells was measured based on ₃H-thymidine incorporation in the presence of increasing doses of irradiated stimulator cells. Autologous and allogeneic PBMCs served as negative and positive stimulator cell controls, respectively. It was observed that human SVF cells elicited a dose-dependent MLR response comparable to that of allogeneic PBMCs (FIG. 6). With progressive passage, the ADAS cells elicited a decreased response that fell to levels comparable to those observed with autologous PBMCs by P1. Immunogenicity of adipose derived cell populations, including human SVF cells and ADAS cells, from multiple donors is shown in Table 5. Positive and negative designations for immunogenicity are based on criteria described in elsewhere herein and are shown for the highest cell dose in each experiment which ranged from 20,000 cells/well (donors 902-917) to 30,000 cells/well (donors 407-611). Based on positive responses for either or both T cell populations, the following populations were immunogenic: SVF cells (4/7 donors), P0 cells (7/9 donors) and P1 cells (4/7 donors). The remaining passaged cell populations (P2-P4) did not induce T cell proliferation in MLR assays with the exception of P2 cells from one donor.

TABLE 5
Immunogenicity of Adipose Derived Cell Populations Assessed
in the MLR Assay Against T Cells Derived from
Two Different Donors.

| | | Adipose Derived Cell Population | | | | | |
|---|---|---|---|---|---|---|---|
| ADAS | T Cell | (20-30K Cells/Well) | | | | | |
| Donor | Donor | SVF | P0 | P1 | P2 | P3 | P4 |
| L040407 | 4 | ND | + | ND | ND | ND | ND |
| | 5 | ND | + | ND | ND | ND | ND |
| L040513 | 4 | ND | + | ND | ND | ND | ND |
| | 5 | ND | + | ND | ND | ND | ND |
| L040519 | 4 | − | + | + | − | − | − |
| | 5 | − | + | − | − | − | − |
| L040608 | 4 | + | − | + | − | − | ND |
| | 5 | + | − | + | − | − | ND |
| L040611 | 4 | + | − | − | − | − | ND |
| | 5 | + | − | − | − | − | ND |
| L040902 | 4 | − | + | − | − | − | − |
| | 5 | − | + | + | + | − | − |
| L040910 | 4 | + | + | − | − | − | − |
| | 5 | + | + | − | − | − | − |
| L040914 | 4 | + | + | + | − | − | − |
| | 5 | + | + | − | − | − | − |
| L040917 | 4 | − | + | − | − | − | − |
| | 5 | − | − | − | − | − | − |

+ = Immunogenic (all 3 criteria described in Methods were satisfied)
− = Nonimmunogenic (≧1 of the 3 criteria described in Methods were not satisfied)
ND = Not Done

Immunosuppression:

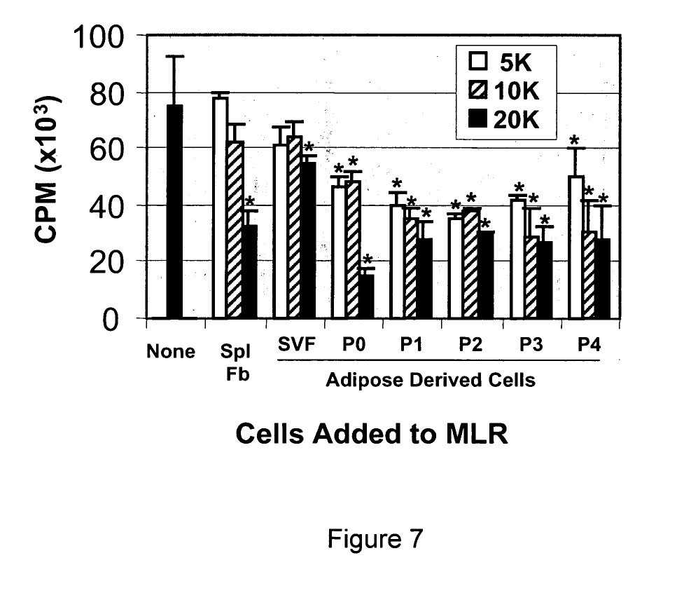

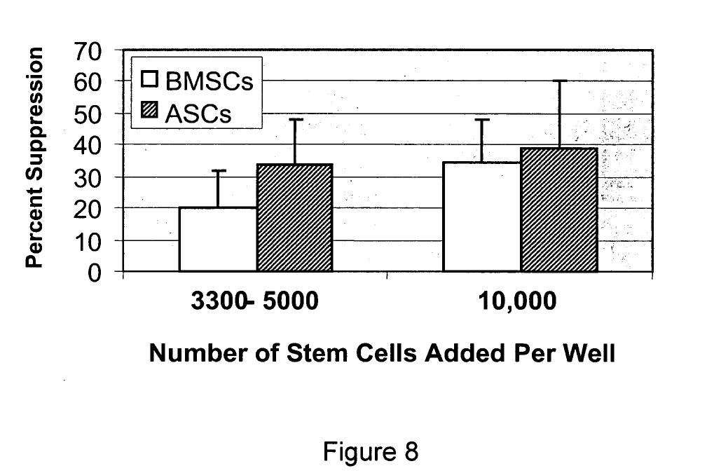

Without wishing to be bound by any particular theory, it is believed that the inability of passaged ADAS cells to stimulate a T cell response may be due to inherent low immunogenicity, to active immunosuppressive mechanisms mediated by the ADAS cells or to a combination of both properties. To determine whether the fat-derived cells were immunosuppressive, they were added to MLR cultures at 5000, 10,000 or 20,000 cells/well. Control MLR cultures either had no cells added or nonsuppressive human splenic fibroblasts were added at the numbers described elsewhere herein to control for suppression due to cell crowding. As shown in FIG. 7, splenic fibroblasts suppressed the MLR cultures only at the highest dose (20,000 cells/well). Using the lower two doses as being valid (no artifactual suppression), significant suppression was mediated by all ADAS cells passages except the SVF population. Percent suppression of the control MLR response (no cells added) mediated by P0-P4 ADAS cells ranged from 33-63%. The results from 4 donors are summarized in Table 6. Percent suppression was determined at the lowest dose of cells (5000 cells/well) since there was no suppression of the MLR at this dose of splenic fibroblasts in any of these experiments. Mean suppression by the SVF population was minimal (10%) whereas suppression by P0-P4 cells averaged 32.0+3.2%. This degree of suppression is significant in view of the low percentage of ADAS cells in these cultures (1.3%). It was of interest to compare the suppressive properties of ADAS cells to BMSCs since BMSCs have similar phenotypic characteristics and differentiation potential as ADAS cells (Gimble et al., 2003 Curr Top Dev Biol 58:137-60). Both cell types suppressed the MLR when added at doses of 3300-10,000 cells/well (FIG. 8). The magnitude of suppression by ADAS cells exceeded that of BMSCs by up to 13%.

TABLE 6
Percent suppression of MLR cultures by adipose derived
cell populations from four different donors.

| ADAS | Adipose Derived Cell Population | | | | | |
|---|---|---|---|---|---|---|
| Donor | SVF | P0 | P1 | P2 | P3 | P4 |
| L040902 | 6.5 | 8.2 | 53.2 | 11.5 | −6.3* | 7.1 |
| L040910 | 13.6 | 22.8 | 14.1 | 38.4 | 38.7 | 42.2 |
| L040914 | 2.8* | 42.7 | 28.3 | −21.7* | 12.3 | 36.2 |
| L040917 | 18.8* | 38.6 | 47.2 | 53.6 | 44.6 | 33.7 |
| Mean | 10 | 28.1 | 35.7 | 34.5 | 31.9 | 29.8 |
| Std Dev | 5 | 15.8 | 17.9 | 21.3 | 17.2 | 15.5 |

*Values not included in means due to poor viability (<50%).

Temporal Changes:

The results presented herein demonstrate that freshly isolated SVF cells can elicit a T cell proliferative response equivalent to that of allogeneic peripheral blood mononuclear cells in a mixed lymphocyte reaction. This immunogenic response declined for early passage (P0, P1) ADAS cells and essentially disappeared for later passage ADAS cells (P2-P4). The immunogenicity of a cell population in the context of alloreactivity is determined primarily by the presence of antigen presenting cells (APCs) within the population. The classic APC is a hematopoietic cell, typically a dendritic cell or macrophage, that expresses MHC class I and class II molecules in addition to costimulatory molecules such as CD80 and CD86. It is noteworthy that the SVF and P0 populations of adipose derived cells, which were found to be immunogenic, contain an APC subpopulation of cells that are most likely monocytes (positive for CD45, CD11a, CD14, CD86 and MHC class II antigens) whereas P1-P4 populations, which did not contain monocytes, were generally not immunogenic. Without wishing to be bound by any particular theory, it is believed that the ADAS cells may alternatively behave as APCs themselves since they express alloantigen (MHC class I antigens) and a number of cell surface molecules which can exhibit costimulatory activity including CD54, CD40, CD80 and CD86. Interestingly, ADAS cells express most of these molecules through at least P4 suggesting that these proteins are not sufficient to endow ADAS cells with APC function or that other mechanisms, such as active immunosuppression, may override immunogenicity. In this study, it has been shown that ADAS cells significantly suppressed T cell proliferation in the MLR. This property was pronounced in P0-P4 cells (mean 32% suppression), but not in the SVF population (mean 10% suppression). To avoid artifactual interpretation of results, i.e., suppression due to cell crowding, suppression experiments were performed at very high ratios of responding cells in the MLR to the test cells (80:1). Control splenic fibroblasts were not suppressive at this ratio. Suppression by ADAS cells was compared to BMSCs since both cell types have similar phenotypic and functional characteristics and BMSCs have been shown to be immunosuppressive by their ability to inhibit T cell proliferation in MLR assays as well as to mitogenic stimulation. Indeed, it was observed that ADAS cells and BMSCs exhibited similar magnitude of suppression. The results presented herein confirm and extend those recently reported by Puissant et al., (2005 Br. J. Haematol. 129:118-29).

BMSCs have been reported to elaborate suppressive molecules, including hepatocyte growth factor and transforming growth factor beta, prostaglandins and indoleamine 2,3-dioxygenase. Several different mechanisms have been proposed to account for BMSC-mediated suppression of lymphocyte proliferation. These include division arrest of activated T cells and B cells by inhibition of cyclin D2 expression, induction of regulatory T cells or APCs, and interference with dendritic cell and cytotoxic T cell maturation. Without wishing to be bound by any particular theory, it is believed that ADAS cells mediate suppression may have similar mechanisms to that of BMSCs. The immunological data presented herein demonstrate that culture-expanded adipose derived cells do not stimulate, but actively suppress alloreactive T cell proliferation demonstrating that these cells can be transplanted across classical histocompatibility barriers. BMSCs have been reported to survive in immunocompetent allogeneic and xenogeneic recipients for longer than expected periods of time. Due to the immunogenic nature of the SVF population, it is likely that transplantation of SVF cells will be limited solely to autologous applications, although manipulation of the graft to remove monocytes may diminish immunogenicity of this population. However, the use of allogeneic ADAS cells as a source of cells for tissue repair or replacement has important implications with respect to the ready availability of adult stem cells for clinical practice and to the practical and commercial aspects of their manufacture and quality assurance.

The results presented herein demonstrate that the characteristics of cells derived from human adipose tissue change as a function of adhesion and expansion in vitro. The stromal vascular fraction cells, isolated by collagenase digestion and differential centrifugation, were heterogeneous with respect to expression of classical hematopoietic markers. Between 8.1% to 17.6% of these initial cells expressed the monocyte/macrophage and pan-hematopoietic antigens CD11a, CD14, CD45, CD86, and HLA-DR. After four successive passages, less than 1% of the adherent adipose derived stem cells expressed CD14, CD45, or CD86 while only 3% or fewer of the cells expressed either CD11a or HLA-DR. These changes in immunophenotype correlated with the level of immunogenicity displayed by the human adipose derived cells in mixed lymphocyte reactions. While the stromal vascular fraction cells and early passage adipose derived stem cells (P0/P 1) elicited a proliferative response from allogeneic T-cells, later passage cells failed to do so. Indeed, the addition of adipose derived stem cells to mixed lymphocyte reactions suppressed the proliferative response of T cells to allogeneic stimulator cells. The results presented herein indicates that it is possible to transplant adipose derived stem cells across traditional histocompatibility barriers.

### Example 3

### Selection of ADAS Cells

The present disclosure demonstrates that ADAS cells express stem cell associated markers including, but not limited to human multidrug transporter (ABCG2) and aldehyde dehydrogenase (ALDH). With respect to ALDH, ALDH is an intracellular enzyme that can be used to select for ADAS cells. Without wishing to be bound by any particular theory, it is believed that a cleavable substrate can be provided to ADAS cells, wherein the substrate when so present in an ALDH+ ADAS cells is cleaved causing the cleaved substrate to signal for the presence of ADLH+ ADAS cells. Such a signal can be in a form of a fluorescence which can be used to sort ALDH+ ADAS cells.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An isolated adipose tissue-derived adult stromal (ADAS) cell exhibiting a non-immunogenic characteristic, wherein said cell has been passaged up to at least the second passage, further wherein said cell expresses a stem cell associated characteristic selected from the group consisting of human multidrug transporter (ABCG2) and aldehyde dehydrogenase (ALDH).

2. The cell of claim 1, wherein said cell has been passaged up to at least the sixteenth passage.

3. The cell of claim 1, wherein exogenous genetic material has been introduced into said cell.

4. The cell of claim 1, wherein said cell is derived from a human.

5. The cell of claim 1, wherein said cell is allogeneic to a recipient thereof.

6. The cell of claim 1, wherein said cell is xenogeneic to a recipient thereof.

7. A method of treating a transplant recipient to reduce in said recipient an immune response of effector cells against an alloantigen, the method comprising: administering to a transplant recipient, an isolated adipose tissue-derived adult stromal (ADAS) cell exhibiting a non-immunogenic characteristic, wherein said ADAS cell has been passaged up to at least the second passage, further wherein said ADAS cell expresses a stem cell associated characteristic selected from the group consisting of human multidrug transporter (ABCG2) and aldehyde dehydrogenase (ALDH), in an amount effective to reduce an immune response of effector cells against an alloantigen, whereby in the transplant recipient said effector cells have a reduced immune response against said alloantigen.

8. The method of claim 7, wherein said effector cell is a T cell.

9. The method of claim 8, wherein said T cell is from a donor and said alloantigen is from a recipient.

10. The method of claim 8, wherein said T cell is from a recipient and said alloantigen is from a donor.

11. The method of claim 8, wherein said T cell is present in the transplant.

12. The method of claim 7, wherein said effector cell is a T cell activated prior to administration of said ADAS cell to a recipient, and further wherein said immune response is the reactivation of said T cell from the donor.

13. The method of claim 7, wherein said ADAS cell is administered to the transplant recipient to treat rejection of the transplant by the recipient.

14. The method of claim 7, wherein said ADAS cell is derived from a mammal.

15. The method of claim 14, wherein said mammal is a human.

16. The method of claim 7, further comprising administering to said recipient an immunosuppressive agent.

17. The method of claim 7, wherein said ADAS cell is administered to the recipient prior to said transplant.

18. The method of claim 7, wherein said ADAS cell is administered to the recipient concurrently with said transplant.

19. The method of claim 7, wherein said ADAS cell is administered as part of the transplant.

20. The method of claim 7, wherein said ADAS cell is administered to the recipient subsequent to the transplantation of the transplant.

21. The method of claim 7, wherein said ADAS cell is administered intravenously to the recipient.

22. The method of claim 7, wherein said effector cell is a cell of the recipient of said donor transplant.

23. The method of claim 7, wherein said ADAS cell is genetically modified.

24. A method of reducing an immune response by an effector cell against an alloantigen, the method comprising: contacting an effector cell with an isolated adipose tissue-derived adult stromal (ADAS) cell exhibiting a non-immunogenic characteristic, wherein said ADAS cell has been passaged up to at least the second passage, further wherein said cell ADAS expresses a stem cell associated characteristic selected from the group consisting of human multidrug transporter (ABCG2) and aldehyde dehydrogenase (ALDH), in an amount effective to reduce an immune response by said effector cell against said alloantigen.

25. The method of claim 24 wherein said effector cell is a T cell.

26. A method of isolating an adipose tissue-derived stromal (ADAS) cell from a population of cells derived from adipose tissue, the method comprising: providing an antibody specific for ABCG2; contacting said population of adipose-derived cells with said antibody under conditions suitable for formation of an antibody-adipose tissue-derived stromal cell complex; and substantially separating said antibody-adipose tissue-derived stromal cell complex from said population of adipose-derived cells; thereby isolating said adipose tissue-derived stromal cell.

27. The method of claim 26, wherein said antibody is conjugated to a physical support.

28. The method of claim 27, wherein said physical support is selected from the group consisting of a microbead, a magnetic bead, a panning surface, a dense particle for density centrifugation, an adsorption column and an adsorption membrane.

29. The method of claim 27, wherein said physical support is selected from the group consisting of a streptavidin bead and a biotin bead.

30. The method of claim 26, wherein said antibody-adipose tissue-derived stromal cell complex is substantially separated from said population of adipose-derived cells using a method selected from the group consisting of fluorescence activated cell sorting (FACS) and magnetic activated cell sorting (MACS).

31. A method of enriching adipose tissue-derived stromal cells from a population of adipose-derived cells, said method comprising: providing an antibody specific for ABCG2; contacting said population of adipose-derived cells with said antibody under conditions suitable for formation of an antibody-adipose tissue-derived stromal cell complex; and substantially separating said antibody-adipose tissue-derived stromal cell complex from said population of adipose-derived cells; thereby isolating said adipose tissue-derived stromal cell.

32. The method of claim 31, wherein said antibody is conjugated to a physical support.

33. The method of claim 32, wherein said physical support is selected from the group consisting of a microbead, a magnetic bead, a panning surface, a dense particle for density centrifugation, an adsorption column and an adsorption membrane.

34. The method of claim 32, wherein said physical support is selected from the group consisting of a streptavidin bead and a biotin bead.

35. The method of claim 31, wherein said antibody-adipose tissue-derived stromal cell complex is substantially separated from said population of adipose-derived cells using a method selected from the group consisting of fluorescence activated cell sorting (FACS) and magnetic activated cell sorting (MACS).

36. A method of identifying an adipose tissue-derived stromal (ADAS) cell positive for ALDH from a population of cells derived from adipose tissue, the method comprising: providing a cleavable substrate specific for ALDH to said population of cells, wherein said substrate when so present in an ALDH+cell is cleaved, further wherein said cleaved substrate emits a fluorescence thereby identifying an ALDH+ADAS cell.
